(19) United States
(12) Patent Application Publication
(10) Pub. No.: US 2015/0191750 A1
(43) Pub. Date: Jul. 9, 2015

(54) PROCESS AND METHOD FOR IMPROVING THE WATER REUSE, ENERGY EFFICIENCY, FERMENTATION AND PRODUCTS OF A FERMENTATION PLANT

(71) Applicants: James R. Bleyer, Maumee, OH (US); Thomas J. Czartoski, Tecumseh, MI (US); Raymond Roach, Midland, MI (US); Jennifer L. Aurandt, Brighton, MI (US)

(72) Inventors: James R. Bleyer, Maumee, OH (US); Thomas J. Czartoski, Tecumseh, MI (US); Raymond Roach, Midland, MI (US); Jennifer L. Aurandt, Brighton, MI (US)

(21) Appl. No.: 14/592,183

(22) Filed: Jan. 8, 2015

Related U.S. Application Data
(63) Continuation-in-part of application No. 13/922,497, filed on Jun. 20, 2013, now Pat. No. 9,029,126.
(60) Provisional application No. 61/662,019, filed on Jun. 20, 2012.

(51) Int. Cl. C12P 7/08; A23K 1/06; C12P 5/02

(52) U.S. Cl. CPC C12P 7/08; C12P 5/023; A23K 1/06

(57) ABSTRACT

A method of improving fermentation, by heating stillage to a temperature of 200 degrees F. to 350 degrees F. resulting in hydrothermally treated stillage, removing from the hydrothermally treated stillage some or all of a composition of suspended solids, dissolved solids, oil, proteins, fiber, or ash, removing some or all of the dissolved solids from the hydrothermally treated stillage by a mechanism of membranes, biological remediation, anaerobic digestion, electro-dialysis, ion exchange, evaporation, gas-stripping, distillation, solvent extraction, or precipitation, using all or a portion of the hydrothermally treated stillage as a component of a media, and using the media for a process of fermentation or biomass production. Metabolites, biomass, media, and hydrothermally treated stillage can be obtained and recovered by this method. Oil, a protein-containing solids fraction, stickwater and a de-oiled stickwater concentrate can be obtained and recovered by this method.

### BACKGROUND OF THE INVENTION

1. Technical Field

The present invention relates to methods of fermentation. More specifically the present invention relates to processing stillage.

2. Background Art

Throughout this application, various publications, including United States patents, are referenced by author and year and patents by number. Full citations for the publications are listed below. The disclosures of these publications and patents in their entireties are hereby incorporated by reference into this application in order to more fully describe the state of the art to which this invention pertains.

Fermentation is the biological process by which sugars and other carbon sources are converted by micro-organisms into metabolites and biomass. For example, ethanol fermentation is the biological process by which sugars are converted to ethanol and carbon dioxide by yeast. Corn and other grains are the main feedstocks used to produce ethanol. Dry milling has previously been used to produce ethanol from grain and other starch sources through fermentation (shown generally in FIG. 1, labeled “Prior Art”). In the case of corn used as the starch source, the corn is milled to flour, slurried, and treated with enzymes to convert the starch to sugars. The sugars are converted to ethanol in large fermenters. The ethanol is recovered through a distillation process. The residual spent grains, referred to as whole stillage, contains corn germ, corn bran, corn oil, unconverted starch, unfermented sugars, yeast cells, yeast metabolites, and other suspended and dissolved solids. The whole stillage stream is generally separated into wet distillers grain (WDG) and thin stillage. The wet distillers grains can be dried to produce Dry Distillers Grain (DDG). A portion of the thin stillage, referred to as backset, is recycled back to the front end of the ethanol process as make up water. The remaining thin stillage is evaporated to syrup, added to the wet distiller's grains and dried as Dried Distillers Grains with Solubles (DDGS). WDG, DDG, and DDGS are important co-products that are critical to the economic viability of the ethanol process. However, their value can be enhanced by extracting more valuable co-products from these streams. It has only recently been a goal to recover additional materials from the co-products for further use.

Prior art exists which addresses the removal of co-products such as germ, protein, and fiber prior to fermentation. Singh, et al. describes a process for recovery of fiber prior to fermentation in a corn dry-grind ethanol process. (Cereal Chemistry 1999, 76(6) 868-872). Khullar, et al. investigated dry and wet fractionation methods for removing germ and pericarp (fiber) prior to fermentation (Cereal Chemistry 2009, 86(6) 616-22). These front-end fractionation processes have not been widely adopted due to inefficiencies associated with the loss of residual starch to the removed fractions, and capital and operating costs. They are nonetheless capable of removing some components prior to fermentation. If ultimately improved and implemented, a front-end fractionation process will still give rise to a stillage stream with valuable fractions of protein and oil. The present invention has utility with stillage produced in a dry-grind plant that practices front-end fractionation.

Materials, such as oil, protein, and other solubles in the whole stillage are very valuable; however, recovery has shown to be inefficient and uneconomical. Recently, various methods have been attempted to recover the additional materials from stillage. These methods include traditional separation techniques such as heating the stillage stream and performing evaporation, using centrifugation, or using membrane filtration, in order to recover these additional materials. The result of each of these separation processes on stillage is a concentrate and a water phase wherein most of the solids have been removed.

A number of methods have been developed involving heat treated stillage for the recovery of fermentation by-products, especially oil. U.S. Patent Application Publication No. 2009/0250412 and U.S. Pat. No. 7,608,729 to Winsness, et al. disclose methods for recovering oil from stillage concentrate including oil resulting from a process used for producing ethanol from corn. Winsness, et al. generally believe that filtration increases operating costs and therefore focus on separation by heating. In one embodiment, the method includes heating the stillage concentrate to a temperature sufficient to at least partially separate, or unbind the oil. The heating step includes heating to a temperature above 212 degrees F. but less than about 250 degrees F. The method also includes the step of pressurizing the heated stillage concentrate to prevent boiling. The method further includes recovering the oil from the treated stillage concentrate using a gravity separation process including centrifugation. The process disclosed by Winsness, et al. does not include treatment of un-concentrated stillage streams. While oil can be recovered from the method of Winsness, et al., there are many products in the thin stillage that are not recovered. For example, the process disclosed by Winsness, et al. does not include recovery of a high solids-high protein fraction and a stickwater fraction (as defined below) nor the improved fermentative value and alternative uses of stickwater. Furthermore, it is generally accepted in the art that heating the thin stillage to higher than 250 degrees F. is harmful to proteins and other biological components.

U.S. Pat. No. 6,106,673 to Walker discloses a process and system for the separation of a fermentation process byproduct into its constituent components and for the subsequent recovery of those constituent components. The process requires 1) mixing a starting mixture containing ethanol byproducts with a liquid (water) to form a diluted mixture, 2) heating of the diluted mixture containing the byproducts so as to separate the oil from a base component (fiber) of the byproduct to which the oil is bound at a temperature from about 140 degrees F. to about 250 degrees F., followed by 3) recovering oil, the base product (fiber), and possibly other substances such as molasses from the mixture. The process can be performed on a large scale and in a continuous fashion using a mechanical separator to recover fibers from the diluted heated mixture to produce a solids stream and a liquor stream and by then removing oil and insoluble substances from the liquor stream in an evaporator assembly. Energy consumption and water consumption are minimized through 1) the use of waste heat from the system's dryer as an energy source for the evaporator assembly and 2) the use of condensed liquids from the evaporator assembly to dilute the mixture. There is no disclosure in Walker '673 of recycle of recovered water or stickwater to fermentation or improvement of fermentation rate or yield by recycle of any or the entire liquor stream to upstream operations.

Thus, while heating and mechanical separation described in prior art provides some separation of co-products, it was not recognized that the use of all or a portion of hydrothermally treated stillage or stickwater can improve fermentation processes.

Thermal hydrolysis has been investigated as a pretreatment step prior to anaerobic digestion of biomass, in particular the anaerobic digestion of waste activated sludge from biological waste water treatment facilities and the pretreatment of cellulosic biomass prior to enzymatic hydrolysis to liberate cellulosic sugars. The former has been commercially implemented while the latter remains a research and development endeavor. Camacho, et al. (Proceedings of the WEFTEC® 2008 Conference, Chicago, Ill. Water Environment Federation) reviewed the use of thermal hydrolysis as a pretreatment to anaerobic digestion of activated sludge and noted the improvements in both sludge dewaterability and biogas yield during anaerobic digestion. Optimal treatment temperatures were generally in the range of 150-200° C. (302-392° F.).

Yu, et al. (Energy & Fuels 2008, 22, 46-60) reviewed the use of hot compressed water (HCW) as a pretreatment for biomass in the production of cellulosic biofuels. The authors focused on the unique physicochemical properties of HCW and the chemistries imparted by HCW as well as the yield of fermentable sugars resulting from enzymatic hydrolysis of the pretreated biomass.

Kim, et al. (including Ladisch) (Bioresource Technology 2008, 99, 5206-5215.) investigated the thermal hydrolysis of distiller's dry grains and solubles (DDGS) from a dry grind ethanol facility as a cellulosic pretreatment prior to enzymatic hydrolysis of the cellulosic biomass. The objective of the thermal treatment of Kim, et al. was to prepare the cellulose of DDGS for downstream enzymatic hydrolysis to glucose by cellulase and beta-glucosidase enzymes. U.S. Pat. No. 5,846,787 to Ladisch, et al. discloses use of thermal hydrolysis in the range of 160-220 degrees C. (320-428 degrees F.) as a pretreatment for cellulosic biomass prior to enzymatic treatment with cellulase.

Other efforts have involved heat treatment and filtration of depleted lignocelluosic fermentation hydrolysate broth to separate undissolved solids from the liquid phase and create a low solids liquid (Hennessey, et al., U.S. Patent Application Publication No. 2012/0178976 and Hennessey, et al., U.S. Patent Application Publication No. 2012/0102823, assigned to Dupont).

It is recognized that the temperatures utilized for hydrothermal pretreatment of biomass prior to cellulosic ethanol fermentation and municipal waste prior to anaerobic digestion are generally greater (300 degrees F.-450 degrees F.) than the preferred range for treating stillage in the present invention (220 degrees F.-300 degrees F.).

Stillage has been investigated for enhancing biological processes. For example, in the prior art ethanol process of FIG. 1, stillage is recycled to the front end as make-up water in the slurry and is referred to as “backset”. The proteins and nutrients in the stillage have been recognized as aiding fermentation; however, this benefit is marginal and the suspended solids in backset limit the amount of fresh grain solids that can be added to fermentation. Therefore, there is a need for treating stillage to increase its value in fermentation and other biological processes.

A number of biological and non-biological methods have been developed for the improvement of thin stillage. Jacob P. Tewalt, et al. in WO2012/122393 assigned to POET Research Inc. disclose a method to clarify thin or whole stillage by growing fungi. Wicking, et al. in U.S. Patent Application Publication No. 2012/2094981 assigned to North American Protein Inc. disclose the use of fungi to remove inhibitory compounds from stillage and create a treated backset having improved ethanol fermentation performance.

J. Van Leeuwen, et al in U.S. Patent Application Publication No. 2010/0196994 assigned to Iowa State University disclose a method of continuous fungi cultivation on thin stillage to produce useful products and remediated water with significantly reduced COD (chemical oxygen demand).

M. Reaney, et al. in U.S. Patent Application Publication No. 2011/0130586, assigned to the University of Saskatchewan, disclose a method of recovering a recyclable water from thin stillage or dewatered (concentrated) thin stillage by polar solvent and/or oil extraction of microbial inhibiting metabolites such as glycerol, lactic acid and 2-phenylethanol (PEA) and the phospholipid α-glycerylphosphorylcholine (GPC) which has potential value in pharmaceutical applications.

J. Jump, et al. in U.S. Pat. No. 7,641,928, assigned to Novozymes North America Inc., disclose the use of enzymes to treat stillage and improve the dewatering properties of stillage.

Prior art processes have tried to remove suspended solids from thin stillage with various flocculating, coagulating or precipitating additives and chemical agents. J. Hughes, et al., in U.S. Pat. No. 8,067,193, assigned to Ciba Specialty Chemicals, discloses the use of anionic polymer additives to increase coagulation and precipitation. D. W. Scheimann and A. S. Kowalski in U.S. Patent Application Publication No. 2006/0006116 assigned to Nalco Company, disclose methods of coagulating and flocculating thin stillage suspended solids using anionic polymer flocculants, cationic coagulants and microparticulate settling aids and removing said suspended solids from the thin stillage. J. Collins, et al. in U.S. Patent Application Publication No. 2012/125859, also assigned to Nalco Co., disclose a method involving ionic flocculants for conditioning and processing whole or thin stillage to aid in the separation and recovery of protein and oil fractions. C. Griffiths in U.S. Patent Application Publication No. 2007/0036881 assigned to Ciba Specialty Chemicals, discloses the removal of suspended solids from thin stillage by treatment with polyacrylamide and electrocoagulation. Verkade, et. al. in U.S. Patent Application Publication No. 2009/0110772 assigned to Iowa State University, describe separating solids from a processed broth through chemical reaction with a phosphorous oxoacid to increase the water solubility of insoluble cellulosic, melaninic, ligninic, or chitinic solids.

Various filtration, microfiltration and ultrafiltration processes have been disclosed in the prior art. Bento, et al. in U.S. Pat. No. 5,250,182 assigned to Zenon Environmental Inc., disclose a step-wise membrane separation process to recover lactic acid and glycerol together, from thin stillage in an ethanol stream. The stepwise process consists of ultrafiltration (UF), nanofiltration (NF) and reverse osmosis membrane units. Demineralized water can be recycled to fermentation or to boiler water make-up feed. Bento et al. suggest that the use of the membrane separation process in the production of ethanol based on the dry-milling of corn, substantially reduces or eliminates the use of a conventional evaporator

Other prior art processes have described removal of solids from the clarified aqueous phase through the use of filters after separation of hot (140-212 degrees F.) concentrated thin stillage into a light oil phase and a heavy aqueous phase and treating the oil phase with alkali chemicals including spent clean in place (CIP) solutions (Woods, et al., U.S. Patent Application Publication No. 2011/0275845, assigned to Primafuel).

None of these biological and non-biological prior art methods for treatment of stillage and solid-liquid separation (with or without benefit of additives) have been shown to improve fermentation by the surprisingly simple process of hydrothermally treating stillage and utilizing the treated stillage as a media component in a fermentation process.

Various methods have been proposed for utilizing stillage for biological purposes other than ethanol fermentation. M. Kriesler and D. Winsness in U.S. Patent Application Publication No. 2010/0028484 assigned to GS Cleantech, disclose methods for producing lipids from various stillage streams by the yeast Rhodotorula glutinis. Kriesler and Winsness also disclose conditioning of the stillage feed stocks by various pre-treatment methods including steam explosion, autohydrolysis, ammonia fiber explosion, acid hydrolysis, sonication and combinations thereof prior to inoculation with the lipid producing micro-organism.

M. Ringpfeil in U.S. Pat. No. 5,981,233 assigned to Roche Vitamins Inc. discloses a process for manufacturing a xylanase enzyme complex from pre-treated thin stillage of rye, where the pretreatment includes removing solids from the rye thin stillage, evaporation of water, adding other nutrient components and autoclaving prior to culturing the enzyme producing organism (Trichoderma).

In summary of the prior art, methods for improving ethanol fermentation, fermentation of other products, or growth of non-alcohol producing microorganisms by addition of stillage which has been hydrothermally treated in the preferred range of 220 degrees F.-300 degrees F. of the present invention has not been described in patents or literature. It has been discovered for the first time that hydrothermally treating stillage and adding the treated stillage to a fermentation process increases fermentation rates and titers. Therefore, it is shown herein that the present invention provides a simple method for improving fermentation by the addition of hydrothermally treated stillage.

While heating and filtration or centrifugation described in prior art provides some separation of co-products, recovery is limited and costs remain high. One advantage of the present invention is that hydrothermal fractionation of stillage produces a physicochemical alteration, which enables a facile separation allowing for improved recovery of co-products. With respect to the present invention, “hydrothermal fractionation” means heating a substantially aqueous stillage stream to a temperature within a prescribed temperature range, and holding at temperature for a period of time within a prescribed residence time range. A saturation pressure is established and maintained during the hydrothermal fractionation step. Physicochemical alteration means that both physical and chemical changes are imparted to the stillage by the hydrothermal fractionation step. Manifest physical changes include changes in the rate of phase separation, relative phase volumetric fractions and phase densities, phase hydrophobicity and changes in color or appearance. Chemical changes include changes in the distribution of non-soluble protein, fat (oil) and carbohydrate (fiber) between the substantially liquid phase and the substantially solids phase. Other chemical changes include solubilization and/or hydrolysis of components to increase the levels of bio-available protein and ammonia in the soluble phase. These physical and chemical changes are mutually dependent and hence the term physicochemical is applied.

Thus heating of stillage has been performed as described in the prior art for recovery of corn oil and other by-products; however, it was not recognized that the hydrothermal treatment of stillage according to the present invention imparts physicochemical changes enabling facile separation into a low solids stickwater fraction, oil and high protein solids fraction. Furthermore and importantly, it will be shown herein that the low solids stickwater fraction provides an enhanced nutrient medium for ethanol and other fermentation processes, thus providing an economic advantage.

Therefore, there is a need for a simple method of producing a physicochemical alteration that changes the co-products in stillage and enables facile separation of co-products in ethanol processing as well as providing streams suitable for improving biological production and recovery of valuable co-products, extracts, metabolites, and treated water.

### SUMMARY OF THE INVENTION

The present invention provides for a method of improving fermentation, by heating stillage to a temperature of 200 degrees F. to 350 degrees F. resulting in hydrothermally treated stillage, removing from the hydrothermally treated stillage some or all of a composition of suspended solids, dissolved solids, oil, proteins, fiber, or ash, removing some or all of the dissolved solids from the hydrothermally treated stillage by a mechanism of membranes, biological remediation, anaerobic digestion, electro-dialysis, ion exchange, evaporation, gas-stripping, distillation, solvent extraction, or precipitation, using all or a portion of the hydrothermally treated stillage as a component of a media, and using the media for a process of fermentation or biomass production.

The present invention provides for metabolites, biomass, and media recovered by this method.

The present invention provides for hydrothermally treated stillage obtained by this method.

The present invention also provides for a method of improving fermentation, by heating stillage to a temperature of 200 degrees F. to 350 degrees F. resulting in hydrothermally treated stillage, removing from the hydrothermally treated stillage some or all of a composition of suspended solids, dissolved solids, oil, proteins, fiber, or ash, adding one or more agents including acids, bases, minerals, organic and inorganic flocculants, polymeric flocculants, microparticulate settling aids, precipitation aids, or salts to assist in the removal of solids, using all or a portion of the hydrothermally treated stillage as a component of a media, and using the media for a process of fermentation or biomass production.

The present invention provides for a method of recovering oil from hydrothermally treated stillage by heating stillage to a temperature of 200 degrees F. to 350 degrees F. and holding for 3-180 minutes resulting in hydrothermally treated stillage, concentrating the hydrothermally treated stillage by removing a portion of the water, and removing oil from the concentrated hydrothermally treated stillage.

The present invention further provides for oil, a protein-containing solids fraction, stickwater and a de-oiled stickwater concentrate obtained by this method.

### DESCRIPTION OF THE DRAWINGS

Other advantages of the present invention are readily appreciated as the same becomes better understood by reference to the following detailed description when considered in connection with the accompanying drawings wherein:

FIG. 1 is a flowchart of a prior art ethanol fermentation process;

FIG. 2 is a flowchart of the hydrothermal fractionation process of the present invention;

FIG. 3 is a flowchart of the hydrothermal fractionation process of the present invention added after separating whole stillage into stillage and wet cake, followed by separation of stickwater and a fraction containing oil and high protein solids from the treated stillage, and processing of stickwater not recycled as backset and the high protein solids fraction through the evaporators and recovering in DDGS;

FIG. 4 is a flowchart of the hydrothermal fractionation process including the optional step of separating whole stillage into stillage and wet cake, followed by separation of treated stillage in a three-phase decanter known as a “tricanter” giving an oil-water emulsion, stickwater, and high protein solids fraction, the oil-water emulsion can be centrifugally separated into oil and additional stickwater, and stickwater not recycled as backset and high protein solids fraction are recovered in DDGS;

FIG. 5 is a flowchart of the hydrothermal fractionation process of the present invention added after separating whole stillage into stillage and wet cake, followed by hydrothermal fractionation of stillage, separation of stickwater from treated stillage and processing of stickwater by biological and/or chemical processing;

FIG. 6 is a flowchart similar to FIG. 5 including biological and/or chemical processing of stickwater and further including dewatering of the high protein solids fraction to produce a second stickwater fraction and dewatered high protein solids for further production of protein meal;

FIG. 7 is a flowchart of the present invention added after separating whole stillage into thin stillage and a first cut solids stream which is forwarded to a particle size reduction step and re-combined with the thin stillage, the combined stream is further separated into second cut solids which are recovered in DDGS and thick stillage which is hydrothermally treated and fractionated into stickwater, oil and high protein solids;

FIG. 8 is a flowchart of the present invention including the steps of whole stillage particle size reduction, solids separation and solids washing prior to hydrothermal treatment of a mixed stream of stillage liquid and wash liquor;

FIG. 9 is a flowchart of the present invention added after separating whole stillage into stillage and wet cake, followed by hydrothermally treatment of stillage, separating the heat treated stillage into a high protein solids fraction containing stickwater and an oil/stickwater emulsion, separating protein from the high protein solids fraction, forwarding the oil/stickwater emulsion to evaporators and recovering oil from a concentrate stream to produce oil and stickwater concentrate, recycling stickwater as fermentation backset and forwarding stickwater concentrate to the DDGS dryer;

FIG. 10 is a flowchart of the present invention, similar to FIG. 9, in which stickwater is used as fermentation backset and stickwater concentrate is further processed by biological or chemical methods and further utilized as a media component for biomass growth or bio-product production;

FIG. 11 is a flowchart of the present invention in which hydrothermally treated stillage is forwarded to an evaporator or series of evaporator stages and a high protein containing solids fraction and an oil fraction are separated from evaporator concentrate;

FIG. 12 is a graph showing the composition of untreated thin stillage and hydrothermally fractionated thin stillage after low G-force separation, FIG. 12 also includes photographs of centrifuge tubes to illustrate the facile separation of hydrothermally fractionated stillage under low-g separation;

FIG. 13 is a chart showing central composite experimental design used in Example 4 to study the effects of time and temperature on hydrothermal fractionation;

FIGS. 14A-14D are graphs of ammonia, soluble (BCA) protein, crude fat and change in suspended solids vs. thin stillage plotted against the reaction severity factor for the designed experiment of Example 4;

FIGS. 15A-15D are graphs of ammonia, soluble (BCA) protein, crude fat, and change in suspended solids versus thin stillage plotted against the reaction temperature for the designed experiment of Example 4;

FIG. 16 is a graph of oil and total suspended solids as percentages of whole stillage for whole, thick, and thin stillage samples prior to hydrothermal fractionation;

FIG. 17 is a semi-log plot of cell counts versus time for growth of Lipomyces starkeyi on stickwater versus thin stillage; and

FIG. 18 is a plot of biogas (methane) production during anaerobic digestion of stickwater.

### DETAILED DESCRIPTION OF THE INVENTION

Most generally, the present invention provides for methods of fermentation that include processing of stillage to improve the overall fermentation process and generate useful products. The present invention provides a method of hydrothermally treating stillage by heating stillage to a temperature of 200 degrees F. to 350 degrees F., altering the physicochemical properties of the stillage, enabling facile separation of the stillage and creating unique product fractions. Preferably, these product fractions include a fraction high in oil, a high protein solids fraction, and a stickwater fraction having low amounts of oil and suspended solids. In addition to low suspended solids and oil content, the stickwater fraction is chemically different than thin stillage of the prior art and can serve as improved backset or media component in a fermentation process.

The present invention also preferably provides for a method of improving fermentation, by heating stillage to a temperature of 200 degrees F. to 350 degrees F. resulting in hydrothermally treated stillage, removing from the hydrothermally treated stillage some or all of a composition of suspended solids, dissolved solids, oil, proteins, fiber, or ash, removing some or all of the dissolved solids from the hydrothermally treated stillage by a mechanism of membranes, biological remediation, anaerobic digestion, electro-dialysis, ion exchange, evaporation, gas-stripping, distillation, solvent extraction, or precipitation, using all or a portion of the hydrothermally treated stillage as a component of a media, and using the media for a process of fermentation or biomass production. Each of these steps are further described below.

“Stillage” as used herein, refers to a cloudy liquid produced during fermentation that includes solids that are not fermentable, solubles, oils, organic acids, salts, proteins, and various other components. Stillage can also contain products of fermentation not separated during the product recovery step. As described in the Background Art, in conventional dry-grind grain ethanol operations the effluent stillage from the bottom of the beer column is known as “whole stillage” which is then separated by centrifugation into “wet cake” and “thin stillage”. Various fermentation processes can produce whole stillage with a wide range of total solids; but values are typically in the range of 11%-18% w/w. In the current ethanol production process, the suspended solids in thin stillage limit the effectiveness of the evaporators, and when recycled as backset, decrease the efficiency of the fermentation process.

“Stickwater” as used herein, refers to a fraction of the stillage stream that is generally very low in suspended solids, typically less than 1 wt % or less than 50% of the suspended solids in conventional thin stillage, and is mainly water and solubles. This term is also further described below.

“Solids washing” as used herein refers to a process to improve the removal of a desired component from a stream such as wet distillers grains or wet cake, such streams comprising up to 60% by weight of dry matter, by mixing or rinsing the solids stream with an aqueous wash liquid and separating the solids to obtain a washed solids stream and a “wash liquor” stream enriched in the desired component.

“Wash liquid” as used herein refers to any aqueous liquid used for solids washing. Preferably the concentration of the component to be removed from the solids is below the carrying capacity of the wash liquid so as to provide a driving force for transfer of the component from the solids to the wash liquid. For example, water, fresh process water, stickwater, thin stillage, de-oiled thin stillage, diluted thin stillage, distillation vapor concentrate, evaporator condensate, dryer vapor condensate, and mixtures thereof are suitable wash liquids for removal of additional oil in a solids washing process. By further example, fresh process water, condensate and treated water are suitable wash liquids for removal of dissolved solids such as salts, minerals, and proteins in a solids washing process.

“High protein solids” as used herein, refers to a fraction of the hydrothermally treated stillage stream that contains greater than 30 wt % of protein on a dry weight basis.

The term “fermentation” as used herein, refers to a biological process, either anaerobic or aerobic, in which suspended or immobilized micro-organisms or cultured cells in a suitable media are used to produce metabolites and/or new biomass.

The term “biological remediation” as used herein, refers to a biological process, either anaerobic or aerobic, in which suspended or immobilized micro-organisms or cultured cells are used to remove dissolved or suspended solids from an aqueous feed stream and thereby improve the clarity and/or chemical quality of said stream. The biological remediation process results in the production of gaseous or liquid metabolites and/or new biomass. Other essential nutrients required by the organisms for viability or acceleration of the remediation process can be provided.

In the prior art, thin stillage is either evaporated and added to dried distiller grains or recycled as backset to the front end of the process. The suspended solids in the portion of the thin stillage that is evaporated cause fouling of heat transfer surfaces. The evaporators must be oversized to account for this fouling. The evaporators must be taken off-line from time to time for cleaning. This adds to the capital cost and operating cost of the fermentation plant.

Thin stillage is less than ideal when used as backset. The suspended solids present in thin stillage backset limits the amount of grain or other carbon source that can be added during the slurry process. Because of the non-fermentable solids in the backset, pumps, heat exchangers, and fermenters must be oversized, increasing the capital cost and operating cost of the process. Furthermore, the suspended solids in the stillage can interfere with the utilization of nutrients during fermentation.

Thin stillage used as backset is also less than ideal because the thin stillage contains glycerol, organic acids and other metabolites. These compounds act as fermentation inhibitors, slowing fermentation and decreasing throughput.

Nevertheless using thin stillage as backset does have some advantages. The soluble proteins from the feedstock and dead cells of the fermentation agent act as nutrients. However, insoluble proteins cannot be utilized. Fermentation plants will operate the stillage centrifuge to maximize overall plant efficiencies which results in thin stillage typically in the range of 1.5%-3% suspended solids (4%-6% total solids). Preferably, when thin stillage is used in prior art processes, it has 4% or less suspended solids. However, the stillage processing method of the present invention creates low solids stickwater that avoids the operational issues associated with higher solids stillage.

In the present invention, processing stillage with higher suspended solids than thin stillage of the conventional process has advantages. It is known that oil is bound to the suspended solids in stillage. By manipulating the solids content of stillage, the present invention can produce a desired protein and oil yield. Processing stillage with higher solids content can also produce a stickwater that is more suitable for use as a fermentation media, increasing product titer. Therefore, the stillage that is processed in the method herein can be whole stillage, typically containing approximately 8-15% suspended solids (11-18% total solids) or a stillage where the total suspended solids are reduced to a level below whole stillage, including reducing solids to the level of thin stillage. Stillage with a suspended solid content less than whole stillage and more than thin stillage is referred to as thick stillage. Thick stillage can have approximately 3 to 8% suspended solids, and preferably between 4 to 8% suspended solids. The solids separation can be done in one or more steps.

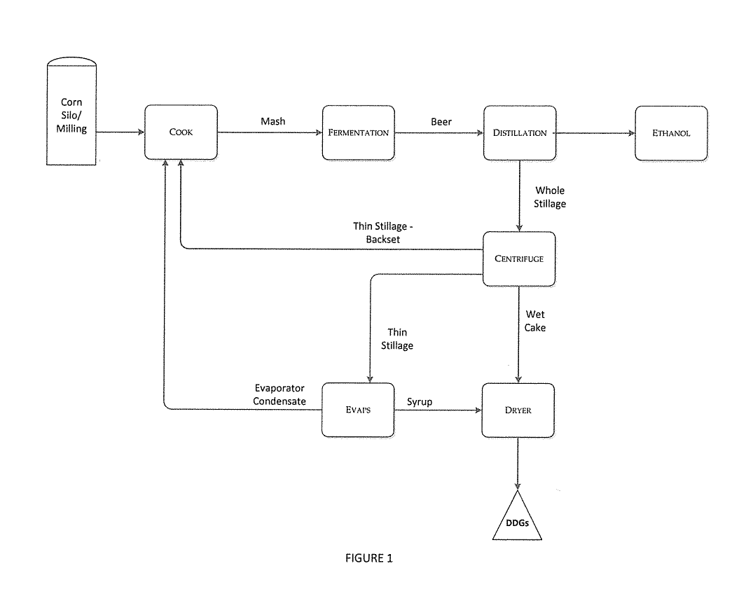

In the method of ethanol fermentation, the grain is milled, slurried, and cooked with enzymes to obtain a sugar-rich mash, fermented to obtain a beer, distilled to produce ethanol, and centrifuged to obtain stillage as shown in FIG. 1. Then, once stillage has been produced, the stillage processing method of the present invention can be introduced into the fermentation process at different points in order to obtain certain products, as further detailed below.

The stillage used in any of the methods herein can be whole stillage, diluted stillage, thin stillage, thick stillage, or concentrated stillage. Wash liquor can also be used as stillage in the present invention. Diluted stillage can include a diluting liquid such as, but not limited to, water, process water, steam, or process vapor and associated process vapor condensate (such as, but not limited to, flash steam, distillation vapor, distillation vapor condensate, evaporated thin stillage vapor, evaporated thin stillage vapor condensate, evaporated stickwater vapor, evaporated stickwater vapor condensate, dryer vapor, or dryer vapor condensate).

Thin stillage can be used in the processing method described in further detail below to generate a stickwater fraction and a high protein solids fraction. Thin stillage is obtained by running the centrifuge under normal operating conditions.

Those skilled in the art appreciate that there are various methods to create a thick stillage stream. After the distillation process, the largest solids (for example, greater than 100 μm) can be removed or separated from the whole stillage by use of a centrifuge, filter, membrane, organic and inorganic flocculants, flocculating polymers, dissolved air flotation, or any other suitable separation method to generate a “large solid wet cake” and a thick stillage. For example, thick stillage can be obtained by running the decanting centrifuge in the conventional ethanol process at reduced speed or for less time than is used to generate thin stillage. By generating a thick stillage by this method, centrifuge operational reliability is enhanced and more oil and other products can be obtained. DDGS are not materially affected by lower productivity as the high protein solids fraction is further separated after hydrothermal treatment and can be combined with wet cake prior to the DDGS dryer.

Thick stillage can also be produced by methods such as removal of water from stillage to concentrate solids, filtration of stillage, centrifugation of whole stillage under centrifuge operating conditions promoting transport of more solids into the centrate, addition of solids to thin stillage, particle size reduction of stillage to increase the suspended solids in the feed to hydrothermal treatment, particle size reduction of grain or a grain slurry to increase the suspended solids in the feed to hydrothermal treatment, and combinations thereof.

Alternatively, thick stillage can also be generated by performing a particle size reduction on all or a portion of the whole stillage stream, before or after separating into wet cake, and combining those reduced particle solids with thin stillage to create thick stillage. Thus the flexibility of the present invention allows for varying solids concentration and stickwater can still be obtained.

Concentrated whole stillage can also be used by the present invention. Various methods can be used to produce concentrated whole stillage, including, but not limited to evaporation and filtration. The evaporation can occur simultaneous with the removal of the target fermentation product. For example, in the production of ethanol, excessive water can be evaporated during the alcohol stripping process, creating a whole stillage stream with a solids content greater than typically created.

Various methods can be used to affect the composition of the stillage prior to hydrothermal treatment to create desired product fractions. For example, whole stillage can be separated into wet cake and thin stillage. The wet cake can be re-suspended with any suitable wash liquid, and separated a second time to further wash and remove dissolved solids and fine suspended solids. The resultant wash liquor can be combined with the thin stillage. The washing step can be combined with the separating step, for example in a washing decanting centrifuge or a washing filtration screen. Therefore, the present invention provides for the steps of washing the wet cake by re-slurrying the wet cake in wash liquid and centrifugally separating a second wet cake and wash liquor, re-slurrying the wet cake in wash liquid and filtering to obtain a second wet cake and wash liquor, or combinations of these steps. Washing of the wet cake can be repeated up to ten times. Separating and washing of the wet cake can be performed in the same device. The stillage can also contain at least a portion of wash liquor separated from washed solids.

Whole stillage can also alternatively be used in the processing method of the present invention and similarly generates a stickwater fraction and a high protein solids fraction. In other words heating the stillage at the temperature described herein, whether whole, thick, or thin, results in a high protein solids fraction and a stickwater fraction with unique properties. The suspended solids content of the stillage can be varied to tailor the desired amount and composition of products in each fraction.

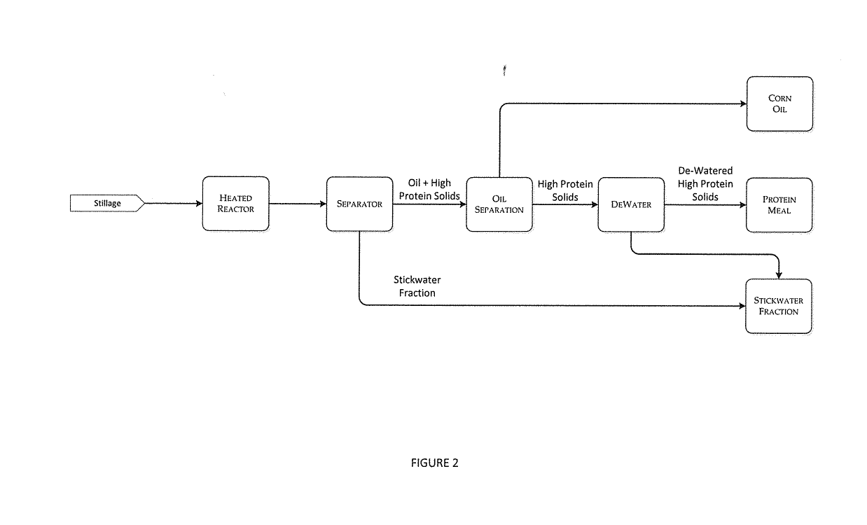

The goal of hydrothermal fractionation is to obtain valuable fractions, reusable water, and improved fermentation media. FIG. 2 shows the main steps of the hydrothermal fractionation method. First, the stillage is heated by a heating mechanism, such as, but not limited to, a heat exchanger or steam injection, to a temperature of 200 degrees F. to 350 degrees F. in, for example, a pressurized reactor. More preferably, the stillage is heated to 220 degrees F. to 300 degrees F. Even more preferably, the stillage is heated to 240 degrees F. to 290 degrees F. The pressure is maintained or held at or above the saturation pressure of the stillage. The stillage is maintained or held at that temperature for 3 to 180 minutes. Afterwards, preferably, the stillage is cooled below its atmospheric boiling point, and preferably below 212 degrees F.

It should be understood that in the case where the stillage has been previously heated to a temperature sufficient for hydrothermal treatment, for example during distillation at above atmospheric pressure, it is not necessary to supply significant additional heat in the hydrothermal reactors. In this case the hydrothermal reactors simply provide sufficient residence time and maintain the target temperature (200 degrees F. to 350 degrees F.) through proper insulation and addition of relatively small amounts of maintenance heat. Therefore, heating can be supplied only as needed to maintain the temperature of fermentation stillage that arrives at the hydrothermal treatment system at the desired target treatment temperature between 200 degrees F.-350 degrees F.

The hydrothermally treated stillage can be added to an operation upstream of a fermentation step. The hydrothermally treated stillage can be cooled by any suitable method prior to use in fermentation media. The present invention also provides for the hydrothermally treated stillage obtained and recovered by methods described herein.

The hydrothermal fractionation step essentially “conditions” the stillage to enable facile separation and creates unique product fractions. These altered fractions cannot be obtained in the prior art processes. Unexpectedly, the stillage can readily separate even under quiescent settling conditions into a high protein solids fraction containing oil and protein solids and a stickwater fraction due to this heating step. The physicochemical change imparted on the stillage by the heating step makes the solids in the stillage less hydrophilic and makes it easier for the stickwater phase to separate from the oil and solids phase. While further mechanical partitioning processes can also be applied as described below, it is unexpected that merely by heating the stillage at this particular temperature range, the stillage can separate into the oil/solids fraction and the stickwater fraction.

As implied by the term physicochemical, the stillage also undergoes chemical changes. Stillage is a complex mixture of yeast cells, proteins, fiber, lipids, minerals, salts, organic acids, glycerol, monosaccharides and oligosaccharides. When used in a fermentation media, many of the components of the stillage would be useful to micro-organisms but are not bio-available. The process of hydrothermal treatment converts or releases these components to increase their bioavailability.

For example, stillage contains many polysaccharides, including residual starch. Not to be bound by theory, it is hypothesized that the hydrothermal treatment of stillage helps to expose residual starch for enzymatic hydrolysis when recycled as backset.

The proteins in stillage are present in tight matrices. These matrices bind the proteins, phosphates, sugars, cations, anions, metals, salts and amino acids. The hydrothermal treatment of stillage unfolds (denatures) and hydrolyzes the proteins in a way that increases the bioavailability of ammonia and soluble proteins.

Stillage also contains oil. Oil present during fermentation adheres to micro-organism cells retarding their ability to convert carbon into biomass or metabolites. The hydrothermal treatment of stillage reduces the corn oil emulsion in stillage making the oil easier to extract. The extraction of oil from stillage reduces the oil in backset and the negative effect on fermentation.

In general, one of the influences on the efficiency of solids separation after hydrothermal treatment step is the degree of solids removal prior to the hydrothermal fractionation step. If stillage with a low suspended solids level is used, the hydrothermal treatment step readily induces separation. If whole stillage is used, the separation does not happen as readily as with thin stillage and whole stillage can therefore require a further mechanical partitioning or separation step as described below. Thus, in general, the heating step makes it easier to release water from solids in the stillage regardless of the type of stillage used. It should also be understood that the heated stillage can directly be used without separating.

Once the stillage has been hydrothermally treated to induce the physicochemical changes, multiple process schemes can be envisioned for separating the treated stillage into various products. These schemes differ based on the type of equipment deployed, the residence time, and the relative g-forces imparted by the specific equipment. Separation can be achieved with a method such as, but not limited to, gravity (quiescent decantation), centrifugation, decanter centrifugation, dissolved air flotation, or any other suitable method. Some examples of separation schemes are provided in the accompanying figures and are described below. Those skilled in the art will recognize that other schemes and equipment options can be utilized to arrive at the desired end-products. Furthermore, it is not necessary to perform all separations and intermediate product compositions can be isolated if desired.

The treated stillage can be separated into a light phase which is substantially oil and a heavy phase which is substantially stickwater and high-protein solids, and the heavy phase can be further separated into a high protein solids phase and a low solids stickwater phase. For example, the separation of the treated stillage into a light phase and heavy phase can be achieved by quiescent decantation for 10 to 180 minutes. The separation of the treated stillage into a light phase and heavy phase can also be performed with a series of centrifugal separators. The further separation of the heavy phase can be performed with a decanting centrifuge.

Alternatively, the treated stillage can be separated into a light phase containing stickwater and oil and a heavy phase containing substantially the high protein solids fraction, and the light phase can be further separated to produce a low solids stickwater fraction and an oil fraction. In this case, the stillage can further be separated into the light phase with a decanter and the light phase can be separated by a centrifuge or quiescent decantation.

The treated stillage can also be separated by performing quiescent decantation to produce a bottom heavy phase which is substantially the low solids stickwater fraction and a top light phase which is substantially high protein solids and oil. This method can further include the step of separating the top light phase into an oil fraction, a high protein solids fraction, and additional low solids stickwater fraction. Any emulsion present in the light phase of quiescent decantation can be separated by an additional centrifugation step.

The treated stillage can be separated into a low specific gravity phase containing oil and high protein solids and a high specific gravity phase containing stickwater by, for example, centrifugal decantation or quiescent decantation. Once the first stickwater fraction has been produced, the low specific gravity fraction can be further dewatered or concentrated. The physical properties of the low specific gravity fraction make it suitable to mechanical dewatering with traditional methods (belt filters, decanters). The de-watered protein/oil fraction can be recovered as a separate product. The low specific gravity fraction containing oil and protein solids can also be concentrated by high speed disk stack centrifugation, dissolved air flotation, evaporation, or any other suitable method. Oil can be weight separated from the protein fraction. The de-oiled high protein stream resulting from the removal of oil can be recovered as a separate product. The dewatered high protein solids can be further processed in evaporators or dryers. Water recovered from dewatering the high protein solids fraction can either be combined with the first stickwater fraction stream or kept as a separate stream. Alternatively, the oil recovery and dewatering step can be combined into a single step using a three-phase decanter or other suitable methods.

The dewatered protein fraction represents a small portion of the total stillage flow, typically 5%-10%, but the high protein content make it valuable. The dewatered de-oiled protein fraction is preferably over 20% solids w/w and more preferably over 25% solids w/w.

The separations as described herein can be performed with a single separation device such as, but not limited to, a three-phase decanting centrifuge (“tricanter”), a three-phase nozzle centrifuge and a three-phase disk stack centrifuge. When a tricanter is used, preferably, the fractions obtained include a high protein solids fraction, a stickwater fraction, and a stickwater/oil emulsion. The stickwater/oil emulsion can be further separated to produce an oil fraction and a second stickwater fraction.

The stickwater fraction of the present invention recovered from the methods herein is of high value and utility. It has very low suspended solids (oil or other solids) content; as low as 1% or less. The stickwater fraction can be recycled to the front end of the plant as enhanced backset to form the slurry, it can also be sent to the evaporators, or any other suitable point in the fermentation process. Since the majority of the solids have been removed from the stickwater fraction used as backset, more sugar, starch or other carbon source can be added to the slurry as compared to when using thin stillage in the slurry, thereby directly increasing the plant's capacity to produce products of fermentation, DDGS, and oil. That portion of the stickwater which is not recycled as backset but is instead forwarded to the evaporators, results in improved evaporator efficiency and operability (less fouling) due to the reduced suspended solids content of stickwater compared to stillage.

Various methods can be used to further process stickwater and the methods and products of the methods included herein are part of the present invention: These include recycling at least a portion of the stickwater as makeup water to a process step upstream of fermentation (such as pre-treatment, slurry, liquefaction, cook, enzymatic hydrolysis, sugar washing and sugar concentrating), filtering at least a portion of the stickwater fraction with membranes or other filtering device, dehydrating at least a portion of the stickwater fraction, concentrating at least a portion of the stickwater fraction, removing glycerol, removing organic acids, removing other organic compounds, removing inorganic compounds such as minerals, metals and salts, adding agents to at least a portion of the stickwater fraction to precipitate components, treating at least a portion of the stickwater fraction and removing fermentation inhibitors, and combinations thereof. Any of the products recovered from these methods are also provided.

The oil of the present invention recovered from the methods herein is also of high value and utility. The oil can be used as an energy source in feed or food products. It can be used directly as or a component of a lubricant. It can be as a feedstock for oleo chemical, biodiesel or renewable diesel.

Thus far, the recovery of oil from hydrothermally treated stillage has been described as occurring prior to or separate from a stillage evaporation step. As has become the convention in the majority of US dry-grind ethanol plants, oil is recovered from evaporator concentrate, typically by centrifugation in the latter stages of a multi-effect evaporator system, the so called mid-evap or post evap oil recovery process. It will be recognized by those skilled in the art that the present invention can be beneficially coupled with a conventional mid-evap/post evap process to increase the amount of recovered oil. Evaporator systems operating on conventional thin stillage typically operate in a temperature range of 150-190 degrees F. across the multiple effects or stages. Such temperatures are useful for unbinding a portion of the oil present in the stillage; however, hydrothermal treatment in the range of 200-350 degrees F. unbinds additional oil and improves the overall oil yield from a mid-evap process. For example following hydrothermal treatment, the treated stillage can be subjected to two-phase centrifugal separation to produce a heavy phase containing high protein solids and some stickwater and a light phase comprising an oil/stickwater emulsion. High protein solids or meal can be recovered from the heavy phase. The emulsion is fed to the evaporators and oil is recovered by centrifugation at an appropriate stage while the end stage yields a stickwater concentrate having reduced suspended solids as compared to conventional syrup produced from non-hydrothermally treated thin stillage. Evaporator fouling is reduced and therefore evaporator capacity enhanced by removal of high protein solids prior to evaporation.

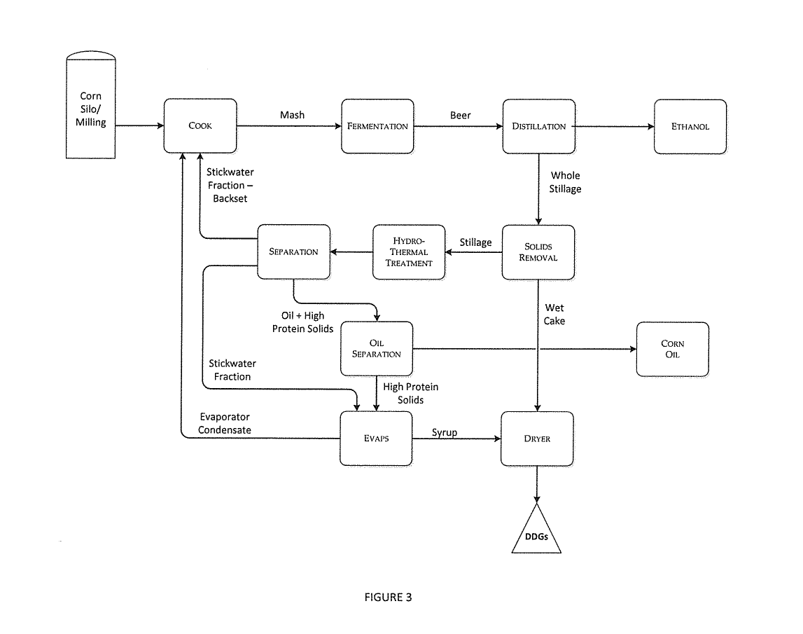

Various process options and recovery methods can be used to influence the composition of stillage and to process and recover products from treated stillage. Some of these options are illustrated in FIGS. 3-11. Many variations of the methods can be used and the figures included herein do not limit the scope of the present invention

One processing option and separation scheme is shown in FIG. 3. Large solids can be removed from the stillage, if desired. The stillage is hydrothermally treated then separated for example by quiescent decantation into a relatively higher specific gravity bottom layer comprising stickwater and a relatively lower specific gravity top layer comprising high protein solids and oil. Oil (such as corn oil) can be removed from the low specific gravity fraction, by for example a centrifugal decanter, and the oil and protein fractions are thereby recovered. Stickwater can be sent to the cook step at the front end of the process as enhanced backset. Some or all of the stickwater and protein fractions can be sent to the evaporators, and separated into the evaporator condensate that is sent back to the cook step, and the concentrated protein fraction that is sent to the dryer as syrup to add to the DDGS.

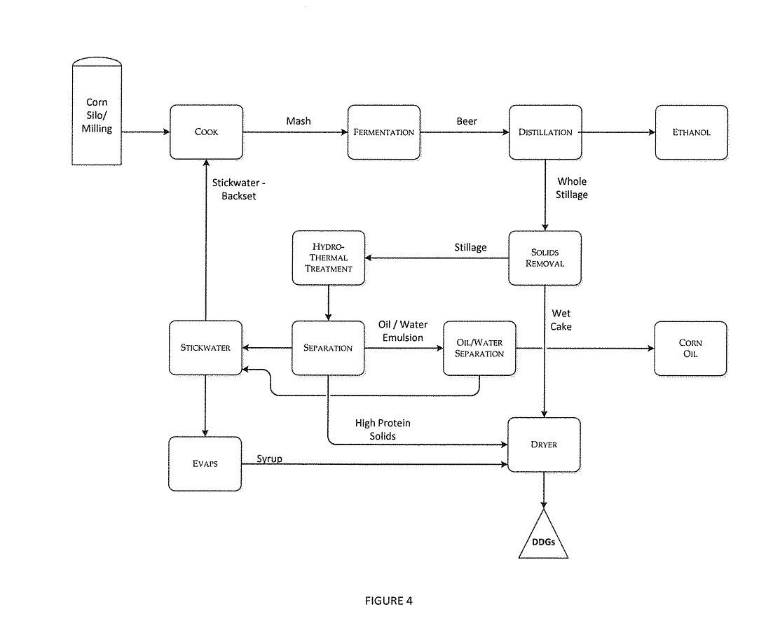

Another processing option and separation scheme is shown in FIG. 4. Stillage is optionally separated into wet cake and stillage and heated in a pressurized reactor. It is then separated into three streams by a three phase decanter (tricanter) or a three phase centrifuge to give cream, liquid, and solids. The cream is an emulsion of oil and water with a small amount of solids. The tricanter liquid stream is mainly stickwater with dissolved solids and low suspended solids. The tricanter solids are mostly suspended solids of fiber and protein. Oil can be separated from the cream, for example, with a high speed centrifuge. The liquid stream (stickwater) can be recycled as backset, evaporated, or used for some other purpose. The solids can be furthered de-watered and/or dried to form a high protein meal. The present invention provides for the high protein meal obtained and recovered from the methods herein.

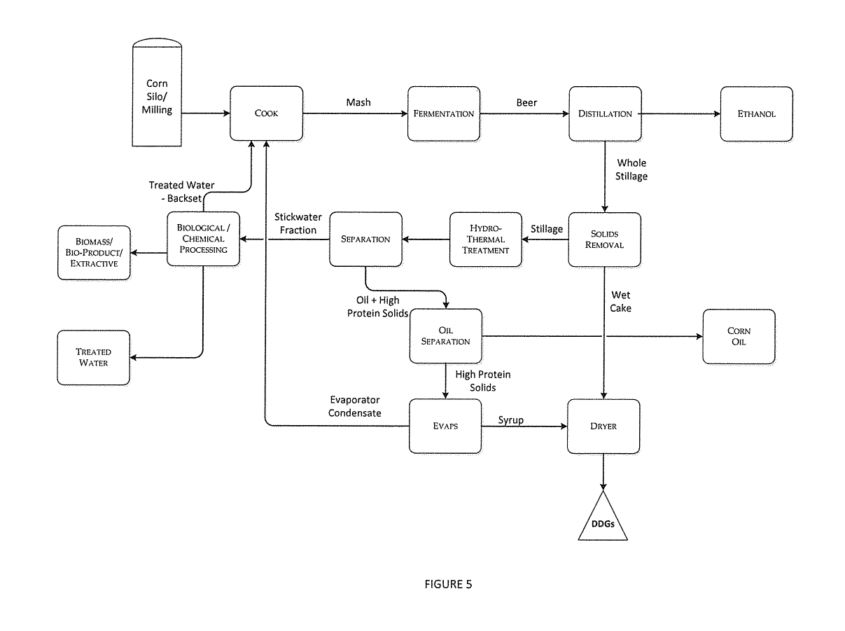

The process shown in FIG. 3 can be further altered so that the stickwater fraction produced by hydrothermal fractionation is sent for further biological or chemical processing as shown in FIG. 5. The stickwater fraction can be treated biologically to further remove fermentation inhibitors and improve its value as a media. Metabolites that are fermentation inhibitors are still present in the stickwater fraction after separation, but without the suspended solids, they can more easily be removed with industrial biological processes such as anaerobic digestion. Anaerobic digestion also produces a methane-rich “biogas” which helps to offset the natural gas usage of the fermentation plant. Presently, anaerobic digestion systems are used to remove dissolved volatile organic compounds such as short chain acids, alcohols and aldehydes from the condensate of thin stillage evaporation.

Alternatively, algae, fungi, or any other suitable microorganisms can be added to the heat treated stillage prior to any of the separation steps or added to stickwater after any of the separation steps. The heat treated stillage or stickwater acts as an improved growth media. Heat treated stillage has multiple advantages as a growth media. The hydrothermal process increases the bioavailability of components as described further below. Components in the heat treated stillage, including ammonia, trace minerals, proteins, and carbohydrates can be used by various micro-organisms. Yeast metabolites, such as glycerol and organic acids, can be used as a carbon source by GMO (genetically modified organism) and non-GMO micro-organisms. These micro-organisms can produce biomass, ethanol or other higher value biofuels or bio-based chemicals. For example, a modified E. coli or yeast can metabolize glycerol to ethanol. After the biological treatment, the stickwater fraction can be recycled or sold.

Stickwater has advantages as a growth media. If the stickwater is used as a media to grow biomass, the reduced suspended solids levels of the stickwater lessen the contamination of the targeted biomass. High levels of suspended solids can interfere with the ability of microorganisms to metabolize dissolved solids in the media.

Stickwater also increases process flexibility. For example, high rate anaerobic digesters and other biological processes often use fluidized bed reactors. Fluid is directed in an upward flow through a bed of microorganisms. The upward force of the fluid and the downward force of gravity keep the bed suspended. If a fluid with high levels of suspended solids is used, the solids can become suspended displacing the microorganisms. The low level of suspended solids in stickwater allows the use of such reactors.

The low solids stickwater can be further processed in order to selectively isolate components. The stickwater fraction can be concentrated by evaporation or membrane separation. Many evaporation process technologies are known in the art. In most dry grind ethanol plants, the evaporation of thin stillage is accomplished in multi-stage (“multi-effect”) evaporator units such as those offered by GEA Wiegand GmbH (www.GEA-Wiegand.com). Each evaporator stage or effect is similar and can be a falling film or forced circulation heat exchanger surrounded by a heating jacket. Liquid recirculation can be provided at each stage. Steam is supplied to the jacket of the first stage where it condenses, the heat of condensation being transferred through the evaporator wall to evaporate a portion of the water from the feed. The first stage concentrate or syrup is forwarded to the second effect evaporator which operates at a lower pressure than the first stage. Heat for the second stage is provided by condensing vapor from the first stage, thus producing second stage vapor and syrup. This process is repeated in subsequent stages and in this manner a large amount of the heat present in the original steam is re-captured for greater overall efficiency. Typically three to five stages are required to concentrate the thin stillage from 6-10% total solids to about 30-40% total solids in the syrup. Evaporation removes primarily water but also small percentages of soluble volatile organic compounds such as short chain alcohols, acids and aldehydes. Anaerobic digestion can then be used to remove these volatile organic compounds from the evaporator condensate.

Membranes can be used to perform ultrafiltration and/or nanofiltration of the stickwater fraction giving a demineralized water stream that is essentially free of dissolved solids and organic compounds larger than membrane pores. Multiple membranes can be used in series. A reverse osmosis (RO) membrane can also be used after the aforementioned filtration steps. Any components isolated by the membranes can be recovered for additional use, such as, but not limited to, lactic acid and glycerol. Additionally, the stickwater can also be chemically treated by addition of acids, bases or other agents to precipitate and recover minerals and salts and/or by addition of solvents to extract metabolites, organic components or plant extractives.

After the biological or chemical processing steps described above, biomass, bio-products, metabolites, and/or extracts can be recovered along with treated water. The treated stickwater can be sold or recycled to the cook step for further use.

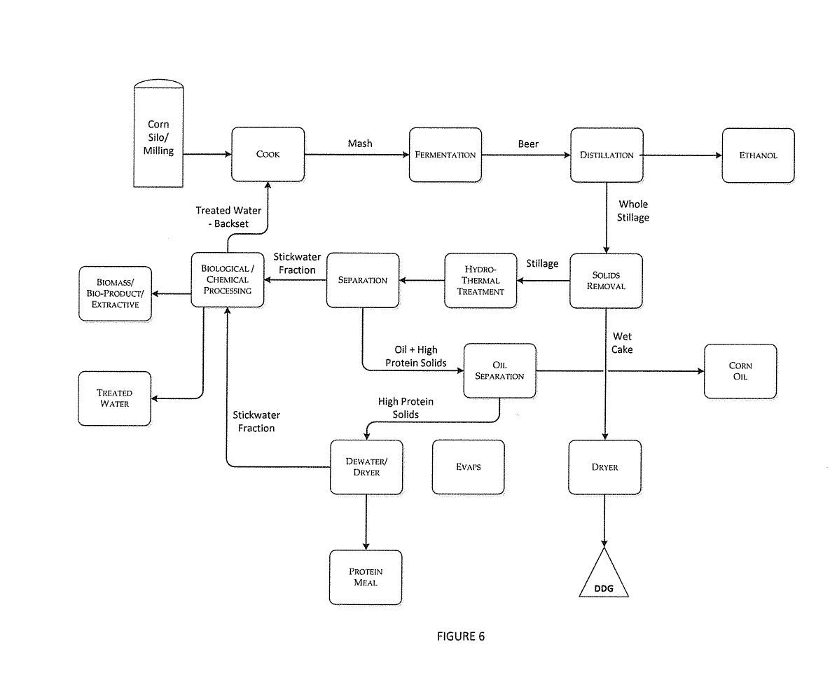

A further process option is shown in FIG. 6, also based on the placement of the hydrothermal fractionation in FIG. 5. In this process, once the protein fraction has been recovered, the protein fraction is dewatered, producing an additional stickwater stream that is sent to the biological or chemical processing step, and a dewatered protein fraction stream that is combined with the wet cake and sent to the dryer to produce dried distillers grains. The treated water from the biological or chemical processing step is recovered and recycled to the front end of the fermentation process. This process eliminates the need for evaporators and reduces cost.

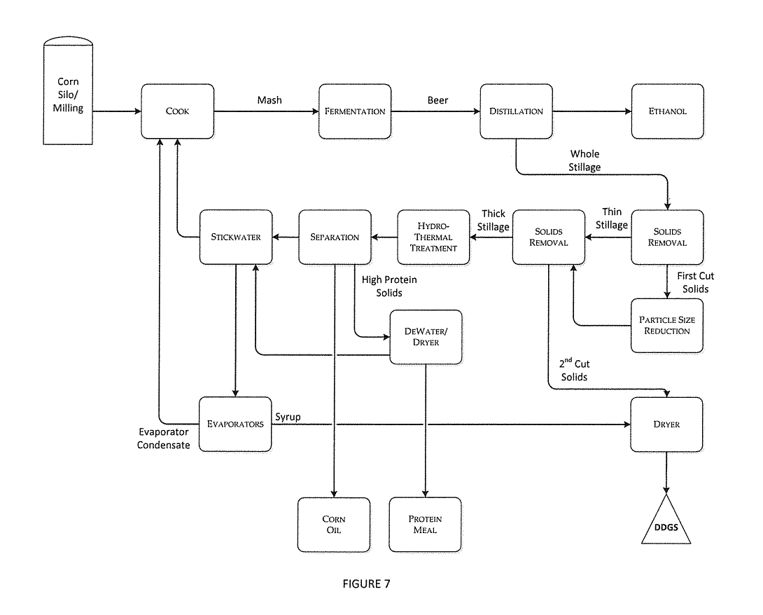

A further process option which exemplifies the use of thick stillage in the present invention is shown in FIG. 7. A solids removal step such as a centrifuge or decanter is applied to whole stillage to obtain thin stillage and a slurry of large particles, denoted as “first cut solids” in FIG. 7. The first cut solids slurry is subjected to a particle size reduction or shear device such as a rotor-stator homogenizer, attrition mill or other such devices known to those skilled in the art. The effluent of the size reduction or shear device can then, for example, be combined with the thin stillage and subjected to a second solids removal step. The fine suspended particles produced in the size reduction step are additive to the suspended particles in thin stillage, thus creating thick stillage. Large particles from the second solids removal step, denoted as “second cut solids” in FIG. 7 are forwarded to a dryer and form the bulk of DDGS solids. The thick stillage from the second solids removal step is hydrothermally treated and separated into stickwater, oil and high protein solids. The dewatered protein fraction can be recovered and dried as a separate product (protein meal) or recovered as DDGS. The use of thick stillage in the present invention provides for additional oil recovery without loss of the enhanced stickwater benefits.

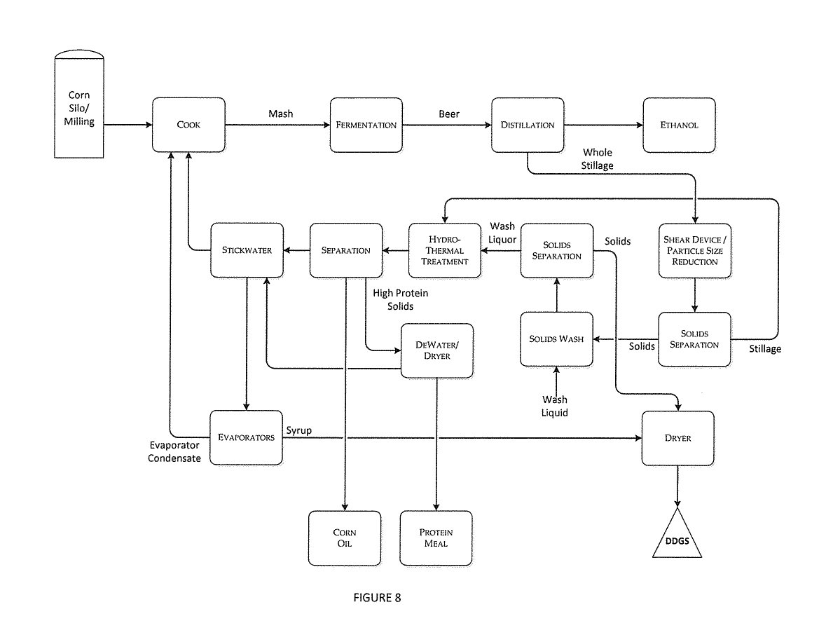

A further process option to influence the composition of the stillage by means of size reduction and solids washing is shown in FIG. 8. The purpose of washing is to liberate additional oil, protein, dissolved solids or fine suspended solids from the wet cake. Whole stillage is subjected to a shear device or particle size reduction device. A solids removal step such as a filter, centrifuge or decanter is applied to whole stillage to obtain thin stillage and wet cake. The wet cake is slurried with any suitable wash liquid. The wet cake slurry is dewatered using any suitable means resulting in a washed wet cake and wash liquor. The wash liquor can be combined with various process streams. It can be combined with the thin stillage as shown in FIG. 8. Alternatively, it can be recycled to the front end of the fermentation process or evaporated. Thin stillage and the wash liquor are subjected, either separately or together, to hydrothermal treatment. The recovery of stillage fractions can occur at various points in the process. The heat treated stillage is separated into an oil/water emulsion and solids stream. Water is removed from the oil/water emulsion by evaporation. Oil is separated from the concentrated oil/water emulsion. The solids stream is separated into dewatered solids and stickwater. The dewatered solids are dried as a protein meal.

Therefore, in one embodiment of the present invention, whole stillage is separated into wet cake and thin stillage. The wet cake is slurried using any suitable liquid and then separated into washed wet cake and wash liquor. Alternatively the wet cake can be washed without resuspension by passing a suitable wash liquid through the formed wet cake such as in a filter, filtering centrifuge or washing centrifuge. The wash liquor can be heated to 200 degrees F. to 350 degrees. In another embodiment, at least one of the following is removed from the wash liquor: oil, water, suspended solids, or dissolved solids. In another embodiment of the invention, at least some of the wash liquor is combined with at least some of the thin stillage either before or after heating. In another embodiment of the invention, the whole stillage is subjected to either a shear device or size reduction device. In another embodiment of the invention, at least some of the wash liquor is used in a fermentation process. In another embodiment of the invention, the wash liquor is evaporated.

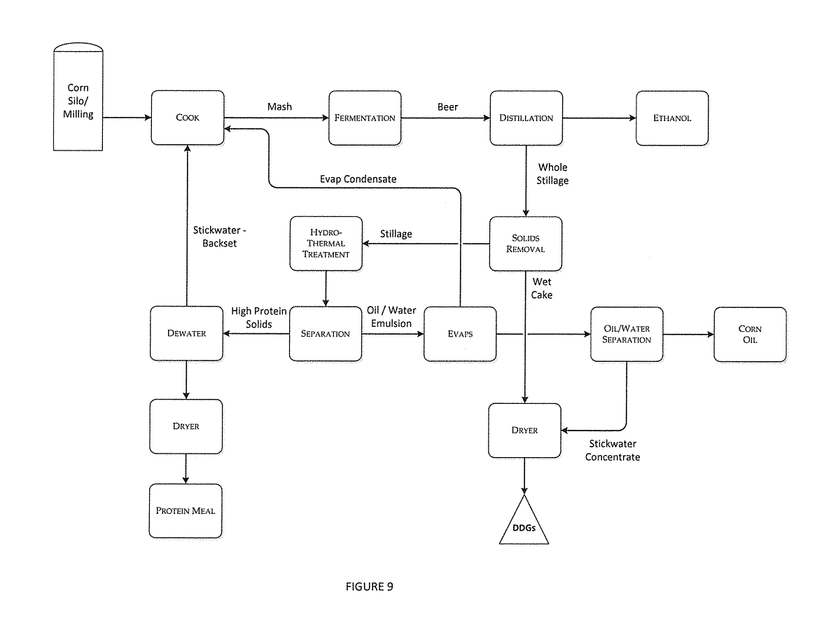

The option to recover oil from hydrothermally treated stillage by way of a mid-evap recovery process is shown in FIG. 9. After separating whole stillage into thin or thick stillage and wet cake, the resulting stillage is hydrothermally treated according to the present invention and separated by for example a two phase nozzle-disk centrifuge into a heavy phase of high protein solids containing a portion of the stickwater and a lighter oil/stickwater emulsion phase. High protein solids and stickwater are produced by dewatering the heavy phase by for example, a high speed decanting centrifuge such as a Sedicanter™ from Flottweg SE (Vilsbiburg, Germany). The dewatered protein fraction is subsequently dried to produce a valuable high protein meal while the stickwater is recycled as fermentation backset. The oil/stickwater emulsion from the two phase nozzle disk centrifuge is fed to a multi-effect evaporator system and a second centrifuge is used to recover oil from an intermediate stage concentrate. The last stage stickwater concentrate is low in oil and lower in suspended solids than concentrated thin stillage and is forwarded to the DDGS dryer.

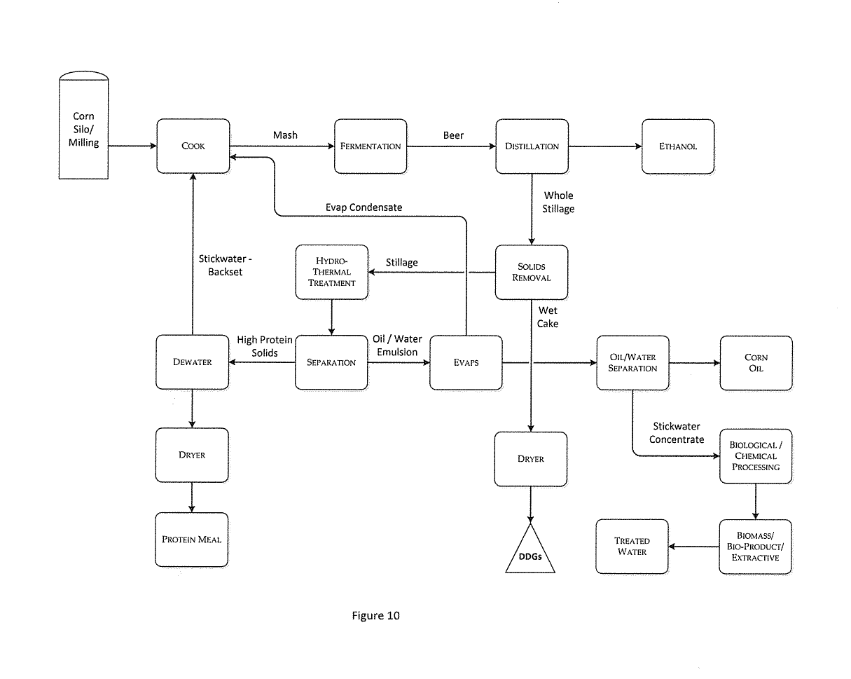

The stickwater concentrate can optionally be treated by a chemical or biological process as shown in FIG. 10. For example, the stickwater concentrate can be treated by anaerobic digestion as described in the context of FIG. 5 resulting in bio-gas and treated water. It will be appreciated by those skilled in the art that stickwater from the protein dewatering step can be combined with stickwater concentrate in any desired ratio prior to the biological and chemical processing depicted in FIG. 10 without compromising the benefits provided by the present invention.

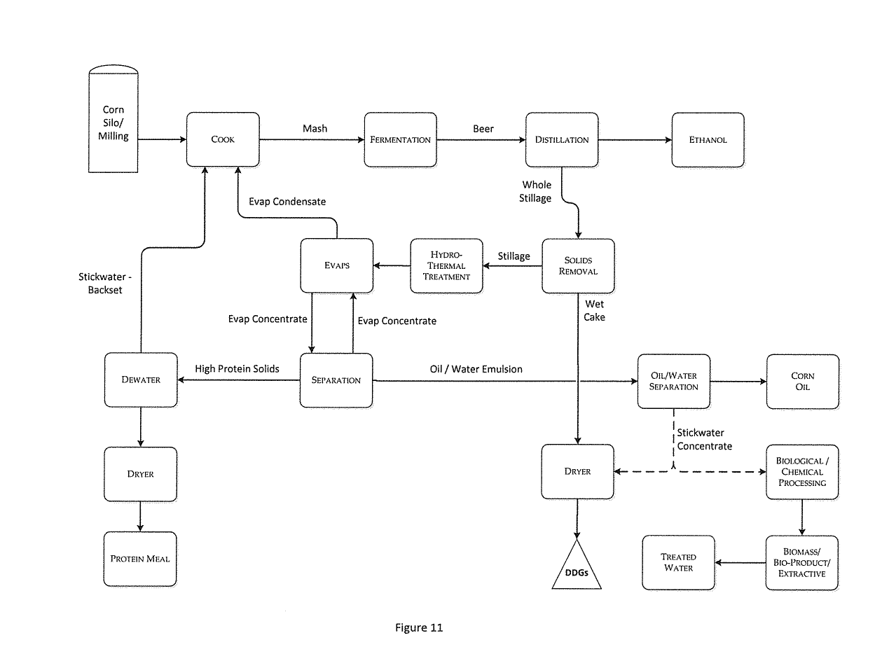

The separation of protein, oil and stickwater from hydrothermally treated stillage can also occur after initiation of an evaporation process as shown in FIG. 11. For example, a concentrate of hydrothermally treated stillage from which a portion of the water has been evaporated can be diverted from one of the stages of a multi-effect evaporator to a nozzle disk centrifuge which separates the concentrate into a heavier phase containing high protein solids and a lighter oil/water emulsion phase. The protein containing phase can be dewatered and dried to produce a protein meal as previously described. The oil/water emulsion can be separated by a second nozzle disk centrifuge into oil and a stickwater concentrate. The stickwater concentrate can be processed by drying or biological and chemical treatment as previously shown in FIGS. 9 and 10.

The present invention provides for a method of performing fermentation by separating whole stillage into stillage and wet cake, hydrothermally fractionating the stillage to create unique product fractions by heating the stillage to a temperature of 200 degrees F. to 350 degrees F., separating the heat treated stillage into a high protein solids fraction, a first stickwater fraction and a stickwater/oil emulsion, recovering oil from the stickwater/oil emulsion, recovering a second stickwater fraction from the stickwater/oil emulsion and adding the second stickwater fraction to the first stickwater fraction, and further processing the first and second stickwater fractions by a process selected from the group consisting of recycling at least a portion of the stickwater to a front end of an fermentation plant, biological processing and chemical processing, and using the first and second stickwater fractions as growth media in said processing step.

The present invention provides for a method of improving fermentation by heating stillage to a temperature of 200 degrees F. to 350 degrees F. resulting in hydrothermally treated stillage, using all or a portion of the hydrothermally treated stillage as a component of a media, and using the media for a process such as fermentation and biomass production.

The fermentation process can produce an alcohol, a metabolite such as organic acids, alcohols, alkanes, olefins, lipids, carbohydrates, proteins, and secondary metabolites or biomass. The fermentation process can be an anaerobic process (e.g. anaerobic digestion) or an aerobic process. The fermentation agent can be algae, bacteria, yeast, fungi, archae, other microorganisms, or cultured cells. The biomass can be algae, bacteria, yeast, fungi, archae, other microorganisms, or cultured cells. Organic compounds in the hydrothermally treated stillage can provide all or a portion of the carbon source. The hydrothermally treated stillage can provide all or a portion of the nutrient requirements. At least one of a carbon source and nutrients can be added to the media. The carbon source can be dextrose, sucrose, fructose, xylose, arabinose, organic acids, glycerol, ethanol, carbon monoxide, carbon dioxide, methane, other alcohols, other carbohydrates, or other hydrocarbons. The carbon source can be derived from cellulosic material.

The method can further include the step of separating the metabolites from the fermentation media, and the step of recovering the biomass from the media. The method can further include the step of using fermentation effluent in additional fermentation processes, such as alcohol fermentation. Biomass and/or metabolites can be recovered prior to the additional fermentation process.

The present invention also provides for metabolites, biomass, and media recovered from the above method.

The method can include the step of removing solids from the hydrothermally treated stillage such as a composition of suspended solids, dissolved solids, oil, proteins, fiber, or ash. The suspended solids can be removed by a mechanism such as centrifuges, decanting centrifuges, filter centrifuge, filters, membranes, hydrocyclone, quiescent decantation, dissolved air floatation, or flocculation. The dissolved solids can be removed by a mechanism such as membranes, biological remediation, electro-dialysis, ion exchange, evaporation, gas-stripping, distillation, solvent extraction, or precipitation. The method can further include the step of adding one or more agents to assist in the removal of solids such as acids, bases, minerals, organic and inorganic flocculants, polymeric flocculants, microparticulate settling aids (diatomaceous earth, bentonite, montmorillonite, colloidal silica borosilicate, or microsand), precipitation aids, and salts. The temperature can also be adjusted to assist in the removal of solids.

If the stillage is thin stillage, some or all of the solids can be removed from the thin stillage prior to or after the heating step. If the stillage if whole stillage, some or all of the solids can be removed from the whole stillage prior to or after the heating step. This method can further include the step of concentrating the stillage prior to treatment.

If the stillage is thick stillage, it can be produced by a method such as removal of water from stillage to concentrate solids, filtration of stillage, centrifugation of stillage under centrifuge operating conditions promoting transport of more solids into the centrate, addition of solids to stillage, particle size reduction of grain or a grain slurry to increase the suspended solids in the feed to hydrothermal treatment, washing of the stillage to promote more solids into the feed, and combinations thereof. Some or all of the solids can be removed from the thick stillage prior to or after the heating step. The method can further include the step of removing some or all of the oil from the stillage before or after the heating step.

The present invention provides for a method of performing fermentation by separating whole stillage into a first cut solids stream and thin stillage, performing a particle size reduction step on all or a portion of the first cut solids, returning the reduced particle size solids to the thin stillage stream to produce thick stillage, hydrothermally treating the thick stillage by heating to a temperature of 200 degrees F. to 350 degrees F., and adding all or a portion of the treated stillage to the fermentation step or an operation upstream of fermentation. Solids can be removed from the stillage after the heating step, by a mechanism such as centrifuges, decanting centrifuges, filter centrifuge, filters, membranes, hydrocyclone, quiescent decantation, dissolved air floatation, or flocculation. Size reduction can be performed with a mechanism such as colloid mills (e.g. ball mills, bead mills), disc mills, pin mills, jet mills, rotor-stator mixers, high-pressure homogenizers, and ultra-sonication. The method can further include the step of performing size reduction on all or a portion of the stillage (such as thin stillage, whole stillage, wet cake, or thick stillage) prior to or after the heating step. Some of the solids can be removed from the stillage prior to or after the size reduction step. The removed solids can be added back to the stillage after particle size reduction.

The present invention provides for a method of performing fermentation by separating whole stillage into wet cake and stillage, hydrothermally treating stillage by heating the stillage to a temperature of 200 degrees F. to 350 degrees F., and adding all or a portion of the treated stillage to the fermentation step or an operation upstream of fermentation.

The present invention provides for a method of increasing bioavailability of stillage components to microorganisms by hydrothermally treating stillage by heating the stillage to a temperature of 200 degrees F. to 350 degrees F., increasing the bioavailability of components in the stillage, using the treated stillage as a media or a component in a media and providing to microorganisms. Increasing the bioavailability of components can include exposing residual starch for enzymatic hydrolysis, unfolding protein matrices, denaturing protein, hydrolyzing protein, and combinations thereof.

The present invention also provides for a method of improving fermentation, by heating stillage to a temperature of 200 degrees F. to 350 degrees F. resulting in hydrothermally treated stillage, removing from the hydrothermally treated stillage some or all of a composition of suspended solids, dissolved solids, oil, proteins, fiber, or ash, adding one or more agents to assist in the removal of solids of acids, bases, minerals, organic and inorganic flocculants, polymeric flocculants, microparticulate settling aids, precipitation aids, or salts, using all or a portion of the hydrothermally treated stillage as a component of a media, and using the media for a process of fermentation or biomass production. Each of these steps can be performed as described above.

The present invention also provides for a method of recovering oil from hydrothermally treated stillage by heating stillage to a temperature of 200 degrees F. to 350 degrees F. and holding for 3-180 minutes resulting in hydrothermally treated stillage, concentrating the hydrothermally treated stillage by removing a portion of the water, and removing oil from the concentrated hydrothermally treated stillage. The hydrothermally treated stillage can be separated into an oil/water emulsion and a protein-containing solids fraction prior to concentration. A protein-containing solids fraction can be separated from the hydrothermally treated stillage after removal of a portion of the water. Oil can be recovered from the oil/water emulsion after concentrating by removing a portion of the water. Concentration can be performed and a portion of the water can be removed from the oil/water emulsion by evaporation. A de-oiled stickwater concentrate can be produced by removing oil from the concentrated emulsion. The present invention also provides for oil, a protein-containing solids fraction, and a de-oiled stickwater concentrate recovered and obtained by this method.

In summary, herein are described the many advantages to the various embodiments of the present invention over prior art processes. First, there are components in the stillage that are fermentation enhancers. For example, the proteins from the carbon source and yeast present in the stillage can potentially supply a source of beneficial amino acids and bio-available nitrogen such as ammonia if properly treated prior to recycle to fermentation. Other insoluble components in the stillage can be fermentation enhancers when solubilized by the present invention. With the stickwater of the present invention, fermentation rates and final titers can be increased.

Second, the present invention can be beneficially incorporated into fermentation plants which practice mid-evaporator or post evaporator oil recovery. Hydrothermal treatment results in greater oil release from stillage and hence, higher oil yields in the mid-evap and post-evap recovery processes. Conversely, production of de-oiled, low suspended solids stickwater prior to evaporation by the present invention potentially allows for the elimination of evaporators. Evaporator condensate that was previously used as make-up water in the cook process can be replaced with additional stickwater. Also, evaporating stillage is energy intensive in prior art processes. Even with the use of multi-effect evaporators, the energy used in evaporation of thin stillage can be as high as 3,000 BTU/gallon of ethanol produced, approximately 10% of all thermal energy used by the plant. Although ethanol plants are highly energy efficient and the energy used in evaporation is recycled to other unit operations, usage minimization or elimination of the evaporators will allow the energy currently utilized for evaporation to be repurposed, such as a Heat Recovery Steam Generator.

Third, recycled stillage can be detrimental to fermentation in prior art processes. The yeast metabolites produced during fermentation and present in the stillage can act as fermentation inhibitors. Examples are glycerol, lactic acid, and acetic acid, among others. The low suspended solids in the stickwater from the present invention allows for more efficient removal of these inhibitors. The present invention provides for the removal of dissolved solids by application of biological treatment, anaerobic digestion, filtration, or other methods. Removal of these inhibitors further enhances stickwater for direct recycle without evaporation.

Fourth, the suspended solids in the stillage are not fermentable and reduce the amount of new feedstock that can be added to the slurry, as fermentation plants typically run at a target total solids target concentration through fermentation to maximize the products of fermentation produced per unit of feedstock processed. By reducing detrimental solids in the backset, hydrothermal fractionation of the present invention can increase plant efficiency and throughput.

Fifth, stillage, if properly treated is an improved growth media for the production of biomass and bio-products. Thus, one additional use of the stickwater fraction is fermentation media for algae, fungi, and other useful microorganisms. The treated stickwater can be sold as a base media base or aqueous feed along with the other bio-products produced instead of or in addition to being recycled back to fermentation.

Sixth, the stillage contains a large portion of oil. In the production of ethanol from corn, the corn oil is up to four times more valuable if extracted than if left in the stillage. However, in stillage of typical fermentation processes, the oil is emulsified in the stillage and does not lend itself to extraction easily. Also, it is impractical and expensive to process the entire flow of stillage to extract the oil. With the process of the present invention, the oil can be extracted with a gravity based separation apparatus. Practicing the present invention on stillage from a corn ethanol plant, between 0.8-1.3 lb of corn oil can be recovered per bushel of corn processed as compared to processes of the prior art where typical yields are 0.4-0.6 lb corn oil per bushel.

Therefore, in summary, the present invention provides for a method of performing fermentation, including the steps of performing the method of hydrothermal fractionation described above, separating and recovering a stickwater fraction, a high protein solids fraction, an oil fraction, and optionally further biologically or chemically processing the stickwater fraction and using the stickwater fraction as growth media. The method can include separating the whole stillage into stillage and wet cake. The method can also include, before separating the whole stillage into stillage and wet cake, the steps of cooking, fermenting, and distilling grain and obtaining a fermentation product. The method can include further affecting the composition of stillage prior to hydrothermal treatment by combinations of shearing and size reduction, solids separation and solids washing steps. The method can include after the further processing step, the steps of recycling some or all of the stickwater to the cook step as enhanced backset, recovering biomass, bio-products, extracts, metabolites, and treated water from the growth media and recycling the treated water. The method can include recovery of oil from hydrothermally treated stillage which is fed to a stillage evaporator system equipped with a mid-evap/post-evap centrifugal oil recovery system. The effluent stickwater concentrate from the evaporator system is amenable to drying or further chemical and biological processing as contemplated for stickwater. The method can further include drying the protein fraction, or if the dewatering step is utilized, the dewatered protein fraction and recovering a high protein meal. Optionally, the protein fraction, or if a dewatering step is utilized, the dewatered protein fraction can be added to the wet cake, and dried, recovering dried distillers grains.

The invention is further described in detail by reference to the following experimental examples. These examples are provided for the purpose of illustration only, and are not intended to be limiting unless otherwise specified. Thus, the invention should in no way be construed as being limited to the following examples, but rather, should be construed to encompass any and all variations which become evident as a result of the teaching provided herein.

### ANALYTICAL METHODS COMMON TO MULTIPLE EXAMPLES

The following analytical methods, shown in TABLE 1, established by AOAC International, were used throughout multiple examples. Other methods are described within specific examples.

TABLE 1

| Analysis | AOAC Method # |
|---|---|
| Dry Weight or Total | 934.01 (24 h, 105 deg C. method) |
| Solids (w/w) | |
| Total Suspended | 934.01 applied to the wet cake of a |
| Solids | sample filtered through 2.2 μm filter |
| | media. |
| Amino Acid analysis: | 994.12 |
| Neutral Fiber | 962.09E (neutral detergent fiber) |
| Crude Protein | 970.09 (Kjehldahl method) |
| Crude Fat/Oil | 920.39C Ether extraction method) |

### Example 1

### Analysis and Comparison of Treatment of Thin Stillage by Invention

Procedures

For the present EXAMPLE 1, thin stillage obtained from a commercial ethanol plant was continuously pumped through a series of Plate and Frame Heat Exchangers (PHEs) into a stirred reactor. The PHEs heated the stillage to 285 degrees F. The reactor's pressure was maintained at the saturation pressure of the stillage. The reactor had a mean residence time of 40 minutes. The conditioned stillage was continuously withdrawn from the reactor and cooled to 185 degrees F., then held in a quiescent decantation tank with a mean residence time of 40 minutes. The relatively high specific gravity stickwater fraction was continuously removed from the bottom of the decantation tank while the relatively low specific gravity fraction containing fat and protein was continuously removed from the top of the decantation tank and collected. The volume ratio of stickwater fraction to fat/protein fraction was 1:1.

Methods of Analysis

The AOAC analytical methods listed above were used in this example.

Results and Discussion

TABLE 2 shows a comparison of thin stillage, stickwater and fat/solids fractions.

TABLE 2

| | Thin | Fat/Protein | Stickwater |
|---|---|---|---|
| | Stillage | Fraction | Fraction |
| Total Solids (w/w) | 8.02 | 8.7 | 6.84 |
| Crude Fat (w/w) | 1.12 | 2.30 | 0.09 |
| Crude Protein (w/w) | 0.99 | 1.18 | 0.65 |

The thin stillage was partitioned into two distinct fractions; a fat/protein fraction and a stickwater fraction. The fat/protein fraction had higher total solids, fat and protein as compared to both thin stillage (8%, 105%, and 19% higher respectively) and stickwater (27%, 2456%, and 82% higher respectively).

### Example 2

### Analysis and Comparison of Low G Separation of Untreated Thin Stillage and Thin Stillage Treated by Invention

Procedures

For the present EXAMPLE 2, untreated thin stillage was obtained from a commercial ethanol plant. The untreated thin stillage was collected at approximately 175 degrees F. Treated stillage was prepared by heating collected thin stillage to 280 degrees F. in a stirred 1-gallon batch reactor, held for 40 minutes at temperature, and then cooled to approximately 175 degrees F. One liter containers of treated and untreated stillage at approximately 175 degrees F. were centrifuged at 400×G for 30 seconds. The samples were then divided volumetrically into a top fraction, middle fraction and bottom fraction, each representing ⅓ of the original sample volume.

Methods of Analysis

The AOAC analytical methods listed above were used in this example

Results and Discussion

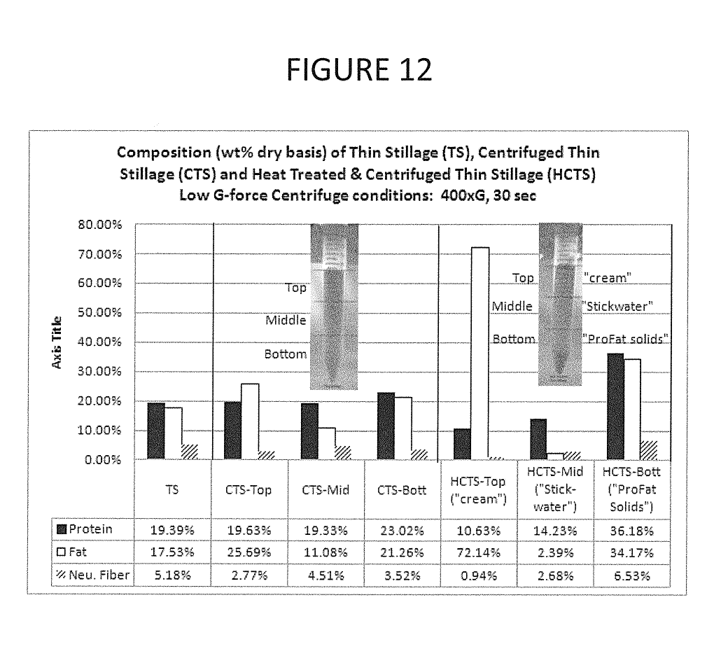

FIG. 12 shows a compositional comparison of the three fractions from the treated and untreated thin stillage centrifuged in 1 liter containers. The photos in FIG. 12 are of the same samples prepared in 15 mL test tubes which provide a clearer visual depiction (than 1 liter bottles) of the formation of sediment (“Solids”) in the treated samples under short duration, low g-force conditions. Sediment was not observed in the untreated sample under low g-force conditions, a further indicator of the facile separation induced by the present invention. The data in FIG. 12 clearly shows that there is no significant partitioning of components top-to-bottom in the untreated centrifuged stillage sample (CTS) while strong partitioning of components occurs in the heat treated and centrifuged thin stillage sample (HCTS) even under low g-force conditions. In particular, the solids fraction is substantially enhanced in fat and protein content relative to thin stillage and the top “cream” portion of the treated sample is likewise enhanced in fat content.

### Example 3

### Fractionation of Low Specific Gravity Stream from Continuous Decantation and Comparison to Thin Stillage and DDGS

Procedures

The low specific gravity stream produced as the upper effluent of a quiescent decantation vessel by the method of EXAMPLE 1, was further fractionated by a tricanter into a second stickwater fraction, an oil fraction and a de-watered de-oiled protein fraction. This final protein fraction was analyzed for dry weight total solids, protein, and oil.

The low specific gravity stream produced by the method of EXAMPLE 1 was pumped at a rate of 3 gpm through an Andritz Decanter Model D3L operating at 3000×G. Oil was collected from the skimmer, the second stickwater fraction was collected as the centrate and the de-oiled de-watered protein fraction was collected as the wet cake.

Untreated thin stillage was also collected and pumped at the same rate through the same decanter at the same settings.

The wet material was then dried in a 105 degrees C. oven overnight and then analyzed for dry weight, protein, and oil.

Methods of Analysis

The AOAC analytical methods listed above were used in this example.

Results and Discussion

The dewatered wet cake of the low specific gravity fraction is compared to the wet cake of dewatered thin stillage in TABLE 3. The low specific gravity fraction easily dewatered in the decanter whereas the thin stillage showed virtually no dewatering. This experiment demonstrated the hydrophobic nature and superior dewatering of the solids processed in accordance with this invention.

TABLE 3
Comparison of Wet Cake from Decanter Dewatering

| | | | Treated Low Specific | |
|---|---|---|---|---|
| | Untreated Thin | | Gravity Stream from | |
| | Stillage | | Quiescent Settling | |
| | Decanter | Decanter | Decanter | Decanter |
| | Feed | Wet Cake | Feed | Wet Cake |
| Total Solids | 4.9 | 4.9 | 9.1 | 24.2 |
| (% w/w) | | | | |

In TABLE 4, the dry weight, protein, fat and neutral fiber analyses for two preparations of the protein fraction (i.e. de-oiled de-watered protein) of the present invention are compared to published data for DDGS. The Protein fraction produced by the present invention has more protein, significantly more fat and significantly less neutral fiber than DDGS.

TABLE 4
Comparison of Protein Fraction to DDGS

| | Protein Fraction | | |
|---|---|---|---|
| | (de-oiled, de-watered) | | |
| | Sample A | Sample B | DDGS^{b} |
| Protein^{a} | 43.4 | 44.1 | 31.2 |
| Fat^{a} | 35.7 | 38.7 | 11.5 |
| Neutral | 1.0 | 0.9 | 42.3 |
| Fiber^{a} | | | |
| Other by | 19.9 | 16.3 | 15.0 |
| difference | | | |

^{a}Expressed as a % of the dry wt.
^{b}Average values from Fastinger and Mahan, (J. Anim. Sci. 84: 1722-1728, 2006) and Stein et al. (J. Anim. Sci. 84: 853-860, 2006) as presented in A. A. Pahm's Ph. D thesis U. of ILL, p. 66 Table 2.1, 2008.

This example illustrates the utility of the invention. A grain fermentation plant can recover a new, high value co-product that is significantly different than the current DDGS co-product. Again, due to the facile separation resulting by heating; proteins, fats, and fibers are obtainable in amounts that would otherwise not be possible to obtain by prior art processes.

### Example 4

### Effect of Time and Temperature on Hydrothermal Fractionation

Procedures

For the present example, a two factor statistical design of experiments (DOE) methodology was used to evaluate the effect of time and temperature on hydrothermal fractionation of thin stillage. The central composite design (CCD) covered the time-temperature ranges of 4-116 minutes and 184 degrees F.-296 degrees F., with a center point at 60 minutes, 240 degrees F. replicated four times. Thin stillage obtained from a commercial ethanol plant was pumped from a well-stirred 5-gallon plastic container through a series of Plate and Frame Heat Exchangers (PHEs) into a stirred 1-gallon batch reactor. The PHEs heated the stillage to the target temperature and the jacketed reactor held the stillage for the prescribed residence time. The reactor pressure was maintained at the saturation pressure of the stillage. At the end of the prescribed residence time, the reactor contents were gravity drained into a clean 1-gallon plastic container, uniformly mixed and poured off into 1-L wide-mouth plastic bottles. The 1-L bottles were centrifuged in a bottle centrifuge (Damon/IEC model EXD centrifuge, Needham Heights, Mass., USA; approx. 18 inch inside chamber diameter) by ramping to full speed (3100 rpm, 2714 G-sec), holding for 1 minute at full speed and ramping down. At the end of centrifugation, the typical top-to-bottom partitioning of material in a full 1-L bottle comprised about 1-2 cm of a floating oil emulsion, about 10 cm of stickwater and about 1-1.5 cm of deposited solids. The oil and water layers from each 1-L bottle were carefully poured off, taking care not to disturb the deposited solids into a 1.25 gallon bench-top gravity decanter (a clear plastic water container of dimensions 12.5 in. length×9 in. height×3 in wide, set on its narrow face at about a 15 degree angle, and equipped with a low point drain valve). The oil and water layers were allowed to gravity separate in the bench-top decanter for 5 minutes after which the bottom stickwater phase was drained through the low-point valve, leaving a small volume of stickwater in the decanter so as to assure a stickwater sample containing no second phase oil. Finally the oil phase was drained from the bench-top decanter with a small amount of residual stickwater. The thin stillage feed and stickwater from each heat treatment condition were analyzed for soluble ammonia, soluble protein, crude protein, crude fat (oil), total solids and suspended solids.

Methods of Analysis

The AOAC analytical methods listed in the table above for crude protein, crude fat, total and suspended solids were used in this example. Specific methods for soluble ammonia and soluble protein are given below.

Soluble protein was analyzed according to the “BCA” method of Smith et al. (Smith, P. K., et al. (1985). Measurement of protein using bicinchoninic acid. Anal Biochem 150:76-85.)

For ammonium determination, the indophenol method according to M. Krom (Analyst 105, 1980, 305-316), a modified Berthelot reaction, was miniaturized as described by C. Laskov et al. (Limnol. Oceanogr.: Methods 4, 2007, 63-71) The reagents were prepared as follows. (A) Buffer solution: In a 1000-mL flask, 33 g potassium sodium tartrate (C₄H₄O₆KNa*4H₂O) was dissolved in 500 mL, then 24 g sodium citrate (C₆H₅O₇Na₃*2H₂O; complexing agent) was added and diluted to 1000 mL. The pH should be controlled and if necessary conditioned to 5.2 by addition of hydrochloric acid. (B) Sodium salicylate solution (phenolic component): 25 g sodium hydroxide (NaOH) was dissolved in 500 mL, then 80 g sodium salicylate (C₇H₅NaO₃) was added and the mixture diluted to 1000 mL. (C) Sodium nitroprusside solution (catalyst): 1 g sodium nitroprusside (Na₂[Fe(CH)₅NO]*2H₂O) was dissolved in 1000 mL deionized water. (D) Sodium dichlorisocyanurate solution (hypochlorite component): 4 g sodium dichlorisocyanurate (C₃N₃O₃Cl₂Na*2H₂O) was dissolved in 1000 mL deionized water.

Solutions B and C were freshly premixed 2:1 (vol/vol) on the day of analysis. Reagent A (400 μL) was added into the microtiter plates, then 240 μL premixed reagent B/C was added, followed by 400 μL sample, and finally, 160 μL reagent D. The microtiter plates were covered and agitated, and after 60 minutes of reaction time, the blue-green indophenol dye was measured at 660 nm.

Results and Discussion

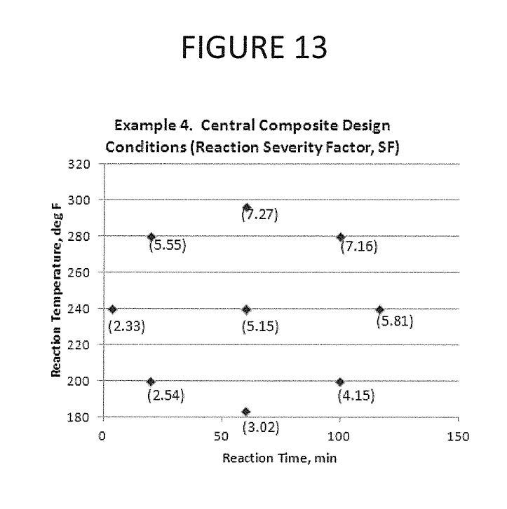

The DOE run conditions are depicted in FIG. 13. In the area of biomass thermal fractionation and lignocellulosic pretreatment, the concept of reaction severity has been applied to account for the combined effects of time and temperature. Overend et al. (Phil. Trans. R. Soc. London A, (1987) 321: 523-536,) developed the generalized severity parameter, Rₒ shown below, where t is the reaction time and w expresses the temperature influence and is related to the average activation energy for hydrolysis reactions. The reaction severity factor, SF is taken as the natural logarithm of the generalized severity parameter and is a unit-less value (S. H. da Cruz et al., J Ind Microbiol Biotechnol (2012) 39:439-447).

$$Ro = {\int }_{0}^{t} exp ( \frac{T - Tref}{ω} ) d t ,$$

which for an isothermal reaction becomes,

$$Ro = exp ( \frac{T - Tref}{ω} ) \times t$$

$$SF = ln ( Ro )$$

T_{ref} was taken as 100 degrees C. (212 degrees F.) and a value of 14.75 was used herein as suggested by Overend et al. for aqueous/steam hydrolysis of biomass. Values for SF, the hydrothermal fractionation run conditions and analytical results are given in TABLE 5.

TABLE 5

| | | | | | | | | % | % |
|---|---|---|---|---|---|---|---|---|---|
| | | | | | | | | Decrease | Decrease in |
| | | | Reaction | | Soluble | | | in Total | Suspended |
| | Reaction | | Severity | | BCA | Crude | Crude | Solids | Solids |
| | Time, | Reaction | Factor, | Ammonia | Protein, | Protein, | Fat | vs Thin | vs Thin |
| | min | Temp, F. | SF | mg/L | g/L | wt % | wt % | Stillage | Stillage |
| Sample | | | | | | | | | |
| Thin | n/a | n/a | 2.53 | 220 | 7.4 | 1.61 | 1.55 | 0.0% | 0.0% |
| Stillage | | | | | | | | | |
| (Feed) | | | | | | | | | |
| Stickwater | | | | | | | | | |
| Samples | | | | | | | | | |
| 1** | 60 | 240 | 5.15 | 255 | 7.4 | 1.61 | 0.58 | 30.8% | 82.2% |
| 2a | 116 | 240 | 5.81 | 265 | 8.6 | 0.97 | 0.47 | 24.3% | 76.1% |
| 2b | 116 | 240 | 5.81 | 279 | 8.5 | 1.10 | 0.5 | 33.8% | 86.7% |
| 3 | 20 | 280 | 5.55 | 283 | 9.4 | 0.9 | 0.66 | 19.8% | 61.7% |
| 4 | 100 | 200 | 4.15 | 215 | 7.3 | 1.11 | 1.12 | 19.8% | 60.3% |
| 5 | 20 | 200 | 2.54 | 218 | 7.7 | 1.06 | 0.82 | 24.8% | 56.9% |
| 6** | 60 | 240 | 5.15 | 264 | 9.4 | 1.04 | 0.61 | 19.6% | 54.8% |
| 7** | 60 | 240 | 5.15 | 255 | 9.1 | 0.92 | 0.58 | 31.9% | 91.6% |
| 8a | 100 | 280 | 7.16 | 315 | 11.1 | 0.95 | 0.17 | 34.9% | 87.5% |
| 8b | 100 | 280 | 7.16 | 315 | 10.2 | 1.08 | 0.11 | 31.7% | 76.9% |
| 9 | 60 | 184 | 3.02 | 240 | 7.9 | 0.82 | 0.08 | 24.3% | 70.0% |
| 10 | 4 | 240 | 2.33 | 247 | 9.0 | 0.82 | 0.76 | 21.0% | 55.4% |
| 11 | 60 | 296 | 7.27 | 317 | 12.1 | 1.01 | 0.17 | 28.9% | 74.5% |
| 12** | 60 | 240 | 5.15 | 265 | 9.6 | 0.83 | 0.59 | 25.0% | 69.1% |
| Ctr Pt Avg | 60 | 240 | 5.15 | 260 | 7.9 | 1.1 | 0.59 | 26.8% | 74.4% |

*For comparison purposes, the time-temperature history and hence R_{SF} for thin stillage was estimated by assuming 35 min at 185 degrees F. as a typical residence time and bottom temperature in the beer column.
**Center points of the DOE replicated four times.

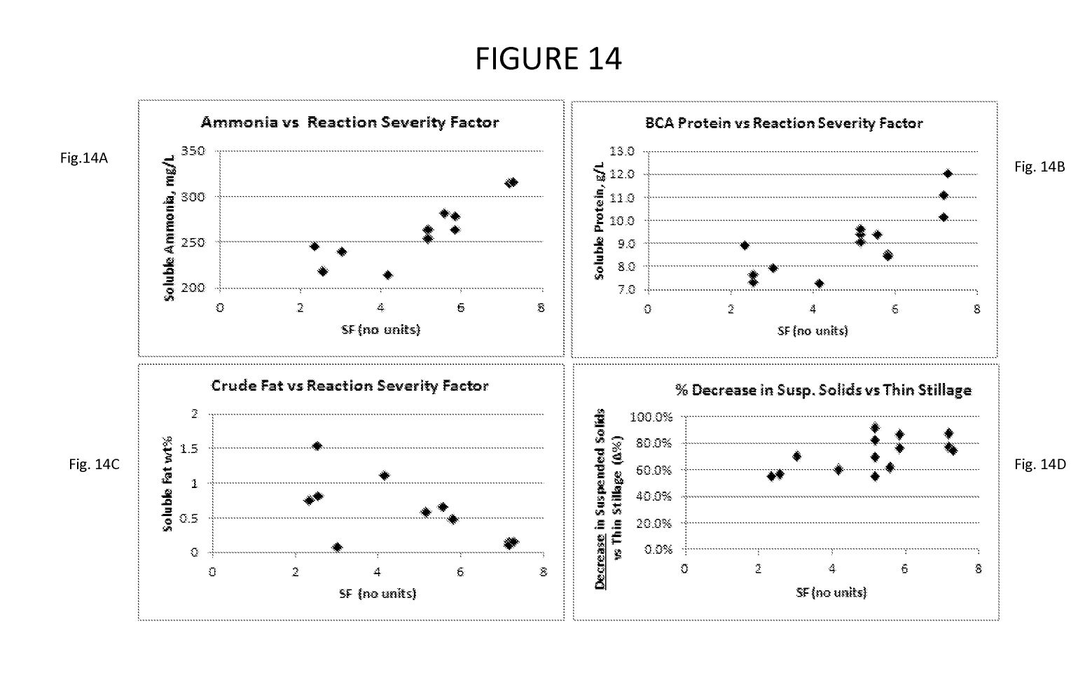

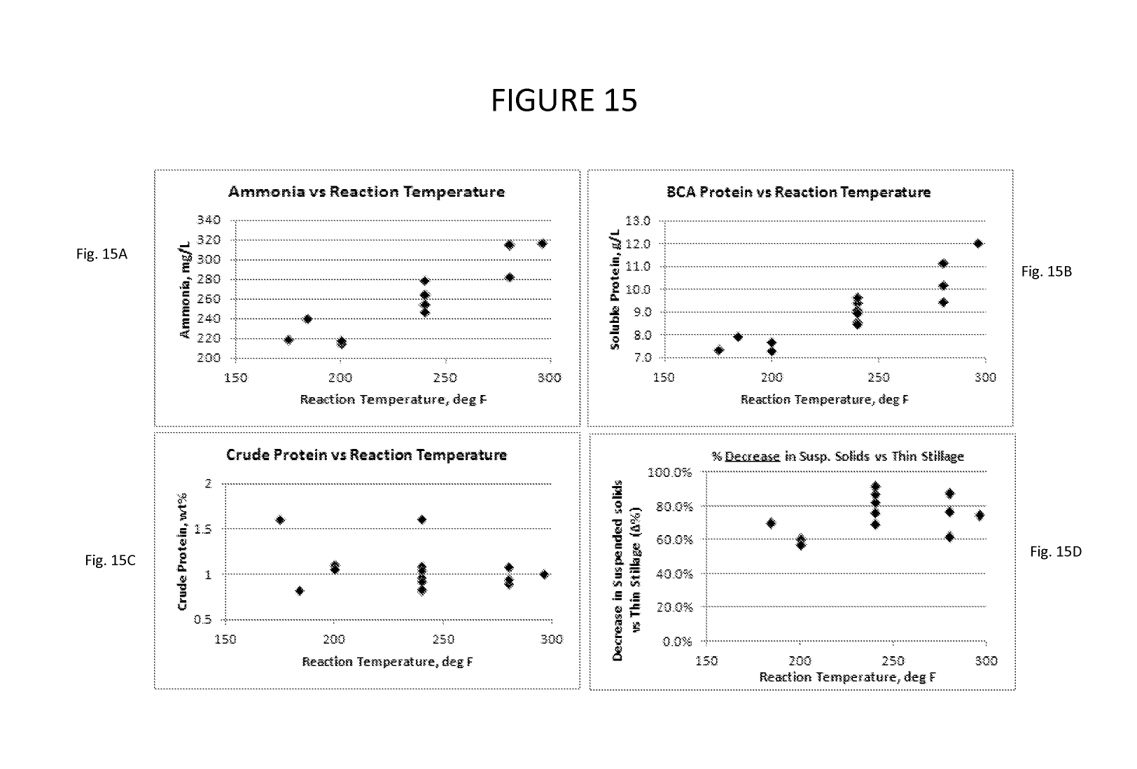

FIGS. 14A-14D show various charts of the data from TABLE 5 plotted against the reaction severity factor, SF while FIGS. 15A-15D show various charts for the data plotted against reaction temperature. Although there is some scatter in the data, ammonia and soluble protein clearly increase in stickwater with increasing reaction severity or temperature. It is believed that hydrolysis reactions are contributing to the observed increases. Ammonia and protein are potential fermentation enhancers when stickwater is recycled as backset. Both crude fat (oil) and suspended solids show a decreasing trend with reaction severity. Since oil is associated with the solids, it is expected that crude fat should decrease as more suspended solids are removed. Hence increasing oil recovery can be expected with increasing reaction severity.

### Example 5

### Continuous Separation of Stickwater, Solids and Oil from Hydrothermally Fractionated Thin Stillage with a Three-Phase Decanter

For the present EXAMPLE 5, thin stillage obtained from a commercial ethanol plant was continuously pumped at a rate of 3 gallons per minute through a series of Plate and Frame Heat Exchangers (PHEs) into a 150 gallon stirred reactor. The PHEs heated the stillage to 250 degrees F. The reactor's pressure was maintained at the saturation pressure of the stillage. The reactor had a working volume of 115 gallons and a mean residence time of 38 minutes. The conditioned stillage was continuously withdrawn from the reactor into a holding tank and pumped at approximately 3 gpm and 150 degrees F. to an Andritz three phase decanter centrifuge (Andritz model D2LC20C PC SA 3PH). Stickwater, oil and high solids fractions were collected. The starting thin stillage and stickwater were analyzed for solids and oil content.

Methods of Analysis

The AOAC analytical methods listed above were used in this example.

Results and Discussion

TABLE 6 shows a comparison of thin stillage and the stickwater fraction. It can be seen that the hydrothermal treatment conditions of 250 degrees F. and 38 minutes and separating the treated thin stillage to separation with a three phase decanter produced a stickwater fraction having low suspended solids and low residual oil.

TABLE 6

| | Thin Stillage | Stickwater |
|---|---|---|
| Total Solids (w/w) | 6.58 | 5.24 |
| Suspended solids | 2.12 | 0.15 |
| (w/w) | | |
| Crude Fat (w/w) | 1.60 | 0.30 |

### Example 6

### Ethanol Fermentation Improved by Stickwater Produced by Hydrothermal Treatment of Thin Stillage at 285 Degrees F.

Dry-grind corn ethanol plants recycle their thin stillage to the front end of the plant to be used as make up water in the cook and fermentation processes. In this example, both thin stillage obtained from a commercial ethanol plant and stickwater prepared thereof were used as the basis for a fermentation medium to which anhydrous glucose was added as a carbon source. No other nutrients were added, thereby showing that the stickwater can be a superior media compared to thin stillage.

Procedures

Stickwater was prepared and collected as in EXAMPLE 1 at a hydrothermal treatment temperature of 285 degrees F.

Culture and Fermentation

The batch fermentations were started with an initial culture of a commercial ethanol producing Saccharomyces cerevisiae (Ethanol Red®, obtained from Fermentis div. of Lesaffre). Two batches of stickwater were produced from commercial thin stillage based on the methods described above, and the resultant stickwater from each batch were then compared to an original sample of thin stillage for ethanol fermentation performance. To a 1 liter sample of thin stillage or stickwater, approximately 200 grams of anhydrous glucose was added as the carbon source and allowed to dissolve. The resultant glucose/sample was added to an autoclaved 1.5 liter stirred reactor (Pyrex® Pro-Culture Spinner Flask (1.5 L); Corning, Corning, N.Y.) and the temperature of the fermentor was equilibrated to 82 degrees F. prior to inoculation.

Inoculum

The inoculum was prepared in a 250 ml sterile Erlenmeyer flask by addition of 1 gram of lyophilized yeast into 100 ml of filter sterilized 2% (w/w) malt extract broth and was incubated at 82 degrees F. for 30 minutes before use. From the inoculum, 5 ml was used to start the fermentations.

Batch Fermentation

An initial sample was taken prior to inoculation and frozen. The fermentation was done at 82 degrees F. with 110 rpm agitation. Fermentation vent locks were fitted to the fermenters at 1 hour after inoculation, to prevent oxygen from entering the vessel. At various time points, samples were removed and frozen prior to analysis via HPLC. After 48 hours the fermentations were stopped.

Methods of Analysis

HPLC analysis for ethanol, glucose (dextrose), and organic acids is based on NREL method LAP 015. Analysis was performed on a Phenomenex Rezex ROA-Organic acid column at 55 degrees C. using 0.005 N sulfuric acid as the eluent and flow rate set at 0.6 ml/min. The detection was via a UV/Vis detector set at 190 nm and CAD (Charged Aerosol Detector). Samples were unthawed, diluted, filtered through a 0.2 micron nylon filter. The injection volume was 20 e was 20 sol Detector). Samples were unthawed, diluted, and filtered through a 0.2 micron nylon filter. The injection volume was 20 μl and the samples were compared against standards.

Results and Discussion

TABLE 7 demonstrates that stickwater provides a superior fermentation medium for ethanol production as compared to thin stillage. Stickwater improved both the rate of ethanol production and yield of ethanol on dextrose versus untreated thin stillage. The theoretical mass yield of ethanol on dextrose is 0.5114 g/g [calculated as 2 mols ethanol*46.068 g/mol)/(1 mol dextrose*180.16 g/mol)=0.5114]. A yield in excess of 0.5114 g/g for both of the treated samples in this example indicates that the stickwater of the present invention provides nutrient value not supplied by untreated thin stillage, thus enhancing the value of stickwater as backset. Additionally this example shows that 100% of the produced stickwater can be recycled as backset without deleterious impact on fermentation performance.

TABLE 7
Ethanol Fermentation results with Stickwater
versus Untreated Thin Stillage

| | | | Average of | Untreated |
|---|---|---|---|---|
| Fermentation | | | Treated | Thin Stillage |
| Metric | Sample A | Sample B | Samples | (Control) |
| Ethanol | 1.97 | 1.95 | 1.96 | 1.67 |
| Production Rate | | | | |
| (g ethanol/l/hr) | | | | |
| Ethanol yield (g/g | 0.517 | 0.529 | 0.523 | 0.435 |
| dextrose utilized) | | | | |
| % of Theoretical | 101.1% | 103.4% | 102.3% | 85.1% |
| Yield | | | | |

### Example 7

### Ethanol Fermentation Improved by Stickwater Produced by Hydrothermal Treatment of Thin Stillage at 240 Degrees F.

In this example, it will be shown that stickwater produced at a temperature of 240 degrees F. provides a beneficial media for ethanol fermentation.

Procedures

The thin stillage feed and resultant stickwater of DOE Condition 2 in Example 4 were used to prepare the fermentation media for this example. The hydrothermal treatment of DOE Condition 2 was for 116 minutes at 240 degrees F. and this condition was replicated twice (2a and 2b) to provide sufficient stickwater for fermentation. Two fermentation runs each were performed with the treated stickwater and thin stillage. All fermentation conditions, preparations and analyses were as described in Example 6.

Results and Discussion

TABLE 8 demonstrates that stickwater prepared by hydrothermal treatment of thin stillage at 240 degrees F. for 116 minutes provides a superior fermentation medium for ethanol production as compared to thin stillage. Additionally this example shows that 100% of the produced stickwater can be recycled as backset without deleterious impact on fermentation performance.

TABLE 8
Ethanol Fermentation results with Stickwater prepared by 240 degrees
F. hydrothermal treatment versus Untreated Thin Stillage

| | | Average of n = 2 |
|---|---|---|
| | | Untreated Thin |
| | Average of n = 2 | Stillage samples |
| | Treated Samples | (Control) |
| Dextrose used, g | 229.5 | 226.4 |
| Ethanol produced, g | 117.3 | 116.7 |
| % Theoretical yield of | 97.5% | 88.5% |
| Ethanol on dextrose | | |
| consumed | | |

### Example 8

### Thin, Thick and Whole Stillage Characterization and Use of Stickwater thereof in Fermentation

In this example, the flexibility of the present invention to produce advantageous stickwater from stillage of varying solids concentrations, i.e. thin stillage, thick stillage and whole stillage, is demonstrated. The advantage of whole stillage or thick stillage, prepared by filtration for example, is that they offer higher recoverable oil concentrations than thin stillage (reference TABLE 9 below).

Procedures

Whole stillage and thin stillage were obtained from a commercial ethanol plant. To produce stillage having a suspended solids concentration between that of whole and thin, whole stillage was filtered through a series nylon filter bags of decreasing pore size (1000, 600, 400, 100 microns). Filtrate from the 100 micron filter was taken as “thick” stillage. Samples of thin stillage, whole stillage, and thick stillage were analyzed for total solids, suspended solids, and % oil. The resultant material was hydrothermally conditioned at 270 degrees F. for 40 minutes, and then separated to produce a stickwater fraction. The stickwater fraction was used as fermentation medium for ethanol production, as previously described in EXAMPLE 6.

Results and Discussion

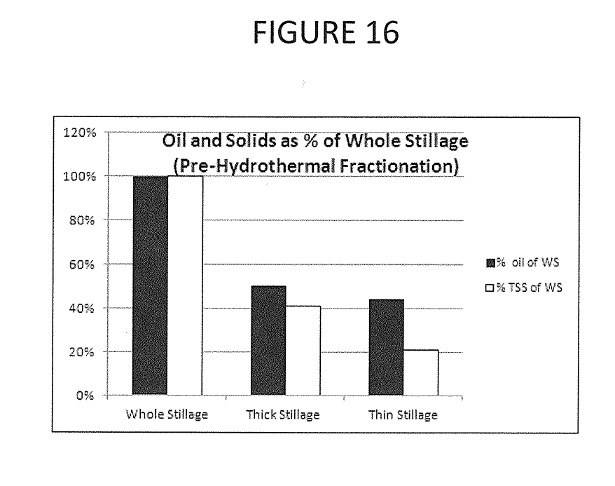

TABLE 9 and FIG. 16 give oil and solids levels prior to hydrothermal treatment and illustrate that a significant percentage of the oil is associated with the suspended solids. Thus, a process which can flexibly treat high and low solids stillage streams will be advantageous. TABLE 10 shows that stickwater prepared by the present invention from any of the stillage concentrations can be used as fermentation media with no loss of performance. The ability to produce stickwater from thin, thick or whole stillage is an unexpected result of the present invention and can provide a producer with greater oil yield, advantageous fermentation yields and process flexibility.

TABLE 9
Oil and Solids prior to Hydrothermal Fractionation

| | Whole | Thick | Thin |
|---|---|---|---|
| | Stillage | Stillage | Stillage |
| Total Suspended | 8.64 | 3.56 | 1.83 |
| Solids (w/w) | | | |
| Pre-Treatment Oil | 1.53 | 0.77 | 0.68 |
| (as w/w % dry basis | | | |
| of Whole Stillage) | | | |

TABLE 10
Ethanol Fermentation using Stickwater prepared from Whole
Stillage, Filtered Whole Stillage and Thin Stillage.

| | Stickwater Source | | |
|---|---|---|---|
| | Whole | Thick | Thin |
| | Stillage | Stillage | Stillage |
| Dextrose Utilized (g/l) | 181.5 | 173.1 | 190.7 |
| Ethanol yield (g/g | 0.430 | 0.455 | 0.435 |
| dextrose utilized) | | | |
| % of Theoretical | 84.1% | 89.0% | 85.1% |
| Yield | | | |

### Example 9

### Analysis and Comparison of Stickwater and Thin Stillage as Fermentation Media for other Microorganisms

In this example, an oleaginous yeast, Lipomyces starkeyi, was chosen as the model microorganism for fermentation. L. starkeyi was chosen due to its ability to grow on a variety of carbon sources and nitrogen sources. Stickwater prepared by the present invention is compared to thin stillage.

Procedures

Stickwater was prepared and collected as in EXAMPLE 1.

Yeast and Fermentation

Both thin stillage and stickwater were sterile filtered through 0.2 micron cellulose acetate membrane prior to inoculation. Lipomyces starkeyi Y-11557 was obtained as ampoules of lyophilized solid from the USDA NRRL culture collection (NRRL, Lab Peoria, Ill.). The inoculum was prepared by adding the full ampoule of lyophilized yeast into a 250 mL sterile shake flask containing 100 mL of filter sterilized 2% malt extract medium and then grown for 24 hours at 25 degrees C. and 110 rpm agitation to produce cells in logarithmic growth phase. The fermentations were performed in sterile 1.5 liter stirred vessels (Pyrex® Pro-Culture Spinner Flask (1.5 L); Corning, Corning, N.Y.) charged with 1 liter of fermentation medium, air flow of 0.95 SLPM, agitation rate of 110 rpm and 80-82 degrees F. A 5 ml inoculum sample was used to start the fermentation and growth was then monitored for 48 hours.

Methods of Analysis

Samples were removed during the course of fermentation and analyzed for microscopic cell count and dry weight (AOAC method). Microscopic cell counts were performed with an Improved Neubauer Counting Chamber using serial dilutions in sterile water as the diluent.

Results and Discussion

In the present example, the stickwater and thin stillage samples were sterile filtered to prevent the potential contamination of the L. starkeyi fermentation batches by foreign micro-organisms. Filtration effectively removed all suspended solids greater than 0.2 μm. Hence the impact of soluble components and any residual ultra-fine suspended solids in the stickwater and thin stillage media is highlighted by this example. TABLE 6 shows the final (48 hours) dry weights of the Lipomyces grown on stickwater versus clarified thin stillage. The total dry weight of the biomass grown on stickwater was 28.6% higher than that grown on clarified thin stillage.

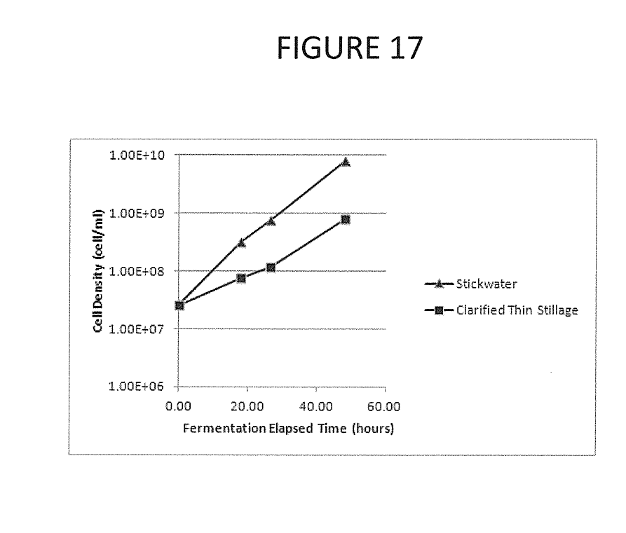

FIG. 17 shows the difference between Lipomyces grown on stickwater versus clarified thin stillage in total cell count. The graph shows that growth on filtered stickwater is much more rapid than growth on filtered thin stillage indicating that the soluble components and any residual ultra-fine suspended solids contained in stickwater provide an advantaged growth medium. Furthermore, this example shows that even fine filtration of thin stillage is not sufficient to provide the unique growth media properties provided by stickwater.

An fermentation plant can diversify its product lines by adding biomass fermentation utilizing stickwater as a medium. An economic advantage is anticipated due to the enhanced growth performance of stickwater versus thin stillage.

TABLE 6
Dry Weight comparison of Lipomyces Starkeyi

| | | Clarified Thin |
|---|---|---|
| | Stickwater | Stillage |
| g/l Dry Weight | 3.01 | 2.34 |

### Example 10

### Removal of Dissolved Solids and Production of Biogas by Anaerobic Digestion of Stickwater

Procedures

For the present EXAMPLE 10, Stillage Stickwater Product was prepared from thin stillage obtained from a local dry-grind ethanol plant according to the methods described in EXAMPLE 4, with hydrothermal treatment conditions of 40 minutes residence time at 250 degrees F. Second phase anaerobic digestion microorganisms were obtained by directly withdrawing a sample from a functioning anaerobic digester operating on evaporator condensate at a local dry-grind ethanol plant. Triplicate stickwater samples were subjected to Biological Methane Potential (BMP) testing using the Automatic Methane Potential Test System (AMPTS) II system and protocol from BioProcess Control Sweden AB. The microorganisms and stickwater were mixed under nitrogen at a 2:1 mass ratio of digester dry solids to stickwater dry solids and placed at 37° C. along with controls that did not contain stickwater. The samples were continuously stirred. The biogas produced was scrubbed with 6M NaOH and the methane produced was determined with an automated and calibrated gas counter. The reactors were allowed to run for 30 days. The methane production was recorded and the methane produced from the microorganisms alone (control) was subtracted from the stickwater methane production. Samples of the initial stickwater and stickwater after 5 days of anaerobic digestion were also analyzed for lactic acid, acetic acid, and glycerol.

Methods of Analysis

Lactic acid, acetic acid, and glycerol were determined by standard methods on a HPLC. A mobile phase of 0.01N H2SO4+5% Acetonitrile with a Rezex ROA organic acid column that is heated to 55° C. was used for analysis. A diode array detector (DAD) was employed and results are compared with commercially available standards.

Results and Discussion

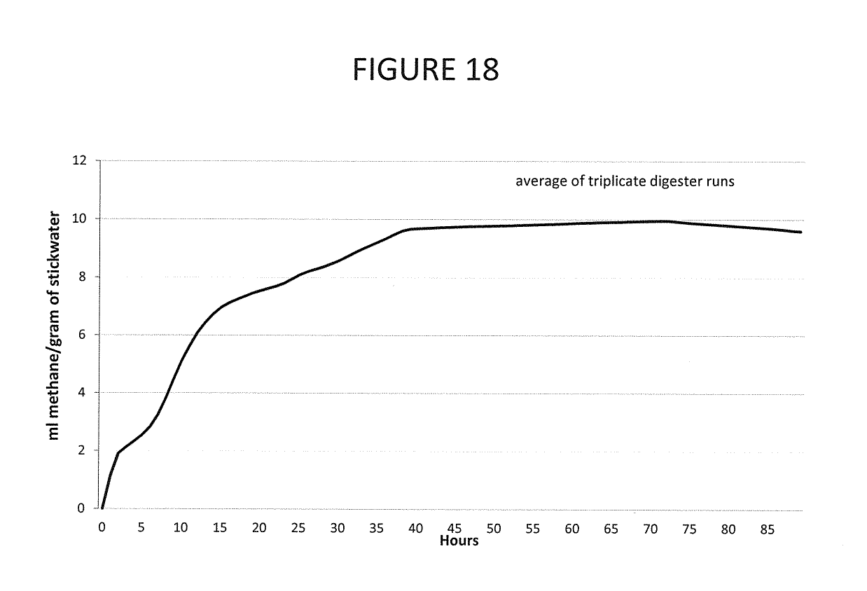

As demonstrated by FIG. 18, the amount of biogas produced was approximately 10 ml/gram of stickwater. Total degradation of acetic acid and glycerol was observed as shown in TABLE 7. This example demonstrated that short chain carbohydrates and fatty acids in stickwater can be completely digested by anaerobic bacteria to produce methane and a clean process stream absent of dissolved fermentation inhibitors.

TABLE 7
Glycerol and acetic acid in stickwater
before and after anaerobic digestion.

| Characterization | Stickwater | AD Stickwater |
|---|---|---|
| Glycerol (% w/v) | 0.78% | 0.00% |
| Acetic acid (% w/v) | 0.06% | 0.00% |

Average of n = 3 digester runs.

The invention has been described in an illustrative manner, and it is to be understood that the terminology, which has been used is intended to be in the nature of words of description rather than of limitation.

Obviously, many modifications and variations of the present invention are possible in light of the above teachings. It is, therefore, to be understood that within the scope of the appended claims, the invention can be practiced otherwise than as specifically described.

What is claimed is:

1. A method of improving fermentation, including the steps of:
heating stillage to a temperature of 200 degrees F. to 350 degrees F. resulting in hydrothermally treated stillage;
removing from the hydrothermally treated stillage some or all of a composition chosen from the group consisting of suspended solids, dissolved solids, oil, proteins, fiber, and ash;
removing some or all of the dissolved solids from the hydrothermally treated stillage by a mechanism chosen from the group consisting of membranes, biological remediation, anaerobic digestion, electro-dialysis, ion exchange, evaporation, gas-stripping, distillation, solvent extraction, and precipitation;
using all or a portion of the hydrothermally treated stillage as a component of a media; and
using the media for a process chosen from the group consisting of fermentation and biomass production.

2. The method of claim 1, wherein said heating step is further defined as holding the stillage at the temperature for 3 to 180 minutes and at a pressure at or above the saturation pressure of the stillage.

3. The method of claim 2, wherein heating is supplied only as needed to maintain the temperature of fermentation stillage which arrives at the hydrothermal treatment system at the target treatment temperature of 200 degrees F.-350 degrees F.

4. The method of claim 1, further including the step of adding the hydrothermally treated stillage to an operation upstream of a fermentation step.

5. The method of claim 1, further including the step of cooling the hydrothermally treated stillage prior to use in fermentation media.

6. The method of claim 1, wherein the fermentation process produces an alcohol.

7. The method of claim 1, wherein the fermentation process produces a metabolite chosen from the group consisting of organic acids, alcohols, alkanes, olefins, lipids, carbohydrates, proteins, and secondary metabolites.

8. The method of claim 1, where the fermentation process is chosen from the group consisting of an anaerobic process and an aerobic process.

9. The method of claim 8, where the fermentation process is anaerobic digestion.

10. The method of claim 1, wherein the biomass is chosen from the group consisting of algae, bacteria, yeast, fungi, archae, and cultured cells.

11. The method of claim 1, wherein organic compounds in the hydrothermally treated stillage provide all or a portion of a carbon source.

12. The method of claim 1, wherein the hydrothermally treated stillage provides all or a portion of the nutrient requirements.

13. The method of claim 1, further including the step of adding at least one of a carbon source and nutrients to the media.

14. The method of claim 13, wherein the carbon source is chosen from the group consisting of dextrose, sucrose, fructose, xylose, arabinose, organic acids, glycerol, ethanol, carbon monoxide, carbon dioxide, and methane.

15. The method of claim 13, wherein the carbon source is derived from cellulosic material.

16. The method of claim 1, wherein the suspended solids are removed by a mechanism chosen from the group consisting of centrifuges, decanting centrifuges, filter centrifuge, filters, membranes, hydrocyclone, quiescent decantation, dissolved air floatation, and flocculation.

17. The method of claim 1, further including the step of adding one or more agents to assist in the removal of solids chosen from the group consisting of acids, bases, minerals, organic and inorganic flocculants, polymeric flocculants, microparticulate settling aids, precipitation aids, and salts.

18. The method of claim 17, wherein the microparticulate settling aid is chosen from the group consisting of diatomaceous earth, bentonite, montmorillonite, colloidal silica borosilicate, and microsand.

19. The method of claim 17, further including the step of adjusting the temperature to assist in the removal of solids.

20. The method of claim 1, wherein the stillage is thin stillage.

21. The method of claim 20, further including the step of removing some or all of the solids from the thin stillage prior to or after said heating step.

22. The method of claim 1, wherein the stillage is whole stillage.

23. The method of claim 22, further including the step of removing some or all of the solids from the whole stillage prior to or after said heating step.

24. The method of claim 1, wherein the stillage is thick stillage.

25. The method of claim 24, wherein the thick stillage is produced by a method chosen from the group consisting of removal of water from stillage to concentrate solids, filtration of stillage, centrifugation of whole stillage under centrifuge operating conditions promoting transport of more solids into the centrate, addition of solids to thin stillage, particle size reduction of stillage to increase suspended solids in the feed to hydrothermal treatment, particle size reduction of grain or grain slurry to increase the suspended solids in the feed to hydrothermal treatment, and combinations thereof.

26. The method of claim 24, further including the step of removing some or all of the solids from the thick stillage prior to or after said heating step.

27. The method of claim 1, further including the step of performing size reduction on all or a portion of the stillage prior to or after said heating step.

28. The method of claim 27, wherein the stillage is chosen from the group consisting of thin stillage, whole stillage, wet cake, and thick stillage.

29. The method of claim 27, further including the step of removing some of the solids from the stillage prior to or after said size reduction step.

30. The method of claim 29, wherein the removed solids are added back to the stillage after particle size reduction.

31. The method of claim 23, further including the step of removing solids from the stillage after said heating step.

32. The method of claim 31, wherein said removing solids step is performed by a mechanism chosen from the group consisting of centrifuges, decanting centrifuges, filter centrifuge, filters, membranes, hydrocyclone, quiescent decantation, dissolved air floatation, and flocculation.

33. The method of claim 27, wherein the step of performing size reduction is performed with a mechanism chosen from the group consisting of colloid mills, disc mills, pin mills, jet mills, rotor-stator mixers, high-pressure homogenizers, and ultra-sonication.

34. The method of claim 1, wherein the stillage is concentrated stillage.

35. The method of claim 1, further including prior to said heating step, the steps of separating the stillage into wet cake and stillage and washing the wet cake with a wash liquid to form washed wet cake and wash liquor.

36. The method of claim 35, wherein the wash liquid is chosen from the group consisting of water, process water, stickwater, thin stillage, de-oiled thin stillage, diluted thin stillage, distillation vapor condensate, evaporator condensate, dryer vapor condensate, and mixtures thereof.

37. The method of claim 35, wherein said washing step is chosen from the group consisting of;
a) re-slurrying the wet cake in wash liquid and centrifugally separating a second wet cake and wash liquor;
b) re-slurrying the wet cake in wash liquid and filtering to obtain a second wet cake and wash liquor; and
c) combinations of a) and b).

38. The method of claim 37, wherein said washing step is repeated up to ten times.

39. The method of claim 35, wherein said separating step and said washing step is performed in the same device.

40. The method of claim 1, wherein the stillage contains at least a portion of wash liquor separated from washed solids.

41. The method of claim 1, wherein the stillage is diluted stillage.

42. The method of claim 41, wherein the stillage is diluted with a liquid or condensable vapor chosen from the group consisting of water, process water, stickwater, steam, flash steam, distillation vapor, distillation vapor condensate, evaporated thin stillage vapor, evaporated thin stillage vapor condensate, evaporated stickwater vapor, evaporated stickwater vapor condensate, dryer vapor, and dryer vapor condensate.

43. The method of claim 1, further including the step of removing some or all of the oil from the stillage before or after said heating step.

44. The method of claim 7, further including the step of separating the metabolites from the fermentation media.

45. The method of claim 1, further including the step of recovering the biomass from the media.

46. The method of claim 1, further including the step of using fermentation effluent in additional fermentation processes.

47. The method of claim 46, wherein the additional fermentation process is alcohol fermentation.

48. The method of claim 46, further including the step of recovering biomass and/or metabolites prior to the additional fermentation process.

49. Metabolites recovered from the method of claim 7.

50. Biomass recovered from the method of claim 10.

51. Media recovered from the method of claim 1.

52. A method of improving fermentation, including the steps of:
heating stillage to a temperature of 200 degrees F. to 350 degrees F. resulting in hydrothermally treated stillage;
removing from the hydrothermally treated stillage some or all of a composition chosen from the group consisting of suspended solids, dissolved solids, oil, proteins, fiber, and ash;
adding one or more agents to assist in the removal of solids chosen from the group consisting of acids, bases, minerals, organic and inorganic flocculants, polymeric flocculants, microparticulate settling aids, precipitation aids, and salts.
using all or a portion of the hydrothermally treated stillage as a component of a media; and
using the media for a process chosen from the group consisting of fermentation and biomass production.

53. A method of recovering oil from hydrothermally treated stillage, including the steps of:
heating stillage to a temperature of 200 degrees F. to 350 degrees F. and holding for 3-180 minutes resulting in hydrothermally treated stillage;
concentrating the hydrothermally treated stillage by removing a portion of the water; and
removing oil from the concentrated hydrothermally treated stillage.

54. The method of claim 53, further including the step of separating the hydrothermally treated stillage into an oil/water emulsion and a protein-containing solids fraction prior to said concentrating step.

55. The method of claim 53, further including the step of separating a protein-containing solids fraction from the hydrothermally treated stillage after said concentrating step.

56. The method of claim 54, further including the step of recovering oil from the oil/water emulsion after said concentrating step.

57. The method of claim 56, wherein said concentrating step is performed by evaporation.

58. The method of claim 57, further including the step of producing a de-oiled stickwater concentrate by removing oil from the concentrated emulsion.

59. Oil recovered by the method of claim 53 or claim 56.

60. A protein-containing solids fraction recovered by the method of claim 54 or claim 55.

61. A de-oiled stickwater concentrate recovered by the method of claim 58.

62. The hydrothermally treated stillage of claim 1.
